(19) United States
(12) Patent Application Publication
(10) Pub. No.: US 2026/0217768 A1
(43) Pub. Date: Jul. 30, 2026

(54) NANOPARTICLE-NHE3 PEPTIDE TO TREAT DIARRHEA

(71) Applicant: The Johns Hopkins University, Baltimore, MD (US)

(72) Inventors: Mark DONOWITZ, Baltimore, MD (US); Jordan J. GREEN, Woodstock, MD (US); Nicholas C. ZACHOS, Columbia, MD (US); Hannah VAUGHAN, Silver Spring, MD (US)

(21) Appl. No.: 19/145,132

(22) PCT Filed: Jan. 8, 2024
(86) PCT No.: PCT/US2024/010681

§ 371 Date: Jul. 1, 2025

Related U.S. Application Data
(60) Provisional application No. 63/478,810, filed on Jan. 6, 2023.

(51) Int. Cl. C07K 14/00; A61K 38/00; A61K 47/69; A61K 49/00; A61P 1/12

(52) U.S. Cl. CPC C07K 14/001; A61K 47/6935; A61K 49/0021; A61K 49/0056; A61P 1/12; A61K 38/00

(57) ABSTRACT

Disclosed are NHE3 mimetic peptides and their conjugates with a reporter molecule or a carboxylated, branched poly(β-amino ester) (PBAE) nanoparticle and their use for treating diarrhea.

### FEDERALLY SPONSORED RESEARCH OR DEVELOPMENT

This invention was made with government support under grants DK089502, DK26523, DK116352, and DK099803, awarded by the National Institutes of Health. The government has certain rights in the invention.

### BACKGROUND

Acute diarrheal diseases are the second most common cause of infant mortality in developing countries. This high rate of mortality is contributed to by a lack of effective drug therapy that shortens the duration or lessens the volume of diarrhea. The epithelial brush border (BB) Na⁺/H⁺ exchanger 3 (NHE3) accounts for a major component of intestinal Na⁺ absorption and is inhibited in most diarrheas. Since increased intestinal Na⁺ absorption can rehydrate diarrhea patients, NHE3 is a potential druggable target for drug therapy for diarrhea.

### SUMMARY

In some aspects, the presently disclosed subject matter provides a peptide having at least about 80% identity with an amino acid sequence selected from SEQ ID NO:1, SEQ ID NO: 2, SEQ ID NO:3, and SEQ ID NO:4, or a fragment thereof. In certain embodiments, the peptide as at least about 81, 82, 83, 84, 85, 86, 87, 88, 89, 90, 91, 92, 93, 94, 95, 96, 97, 98, 99 or 100% identity with an amino acid sequence selected from SEQ ID NO: 1, SEQ ID NO: 2, SEQ ID NO:3, and SEQ ID NO:4, or a fragment thereof. In particular embodiments, the peptide comprises SEQ ID NO: 1 or SEQ ID NO:2. In more particular embodiments, the peptide comprises SEQ ID NO:2.

In other aspects, the presently disclosed subject matter provides a conjugate of the peptide having an amino acid sequence selected from SEQ ID NO:1, SEQ ID NO:2, SEQ ID NO: 3, and SEQ ID NO:4, or a fragment thereof, wherein the peptide is conjugated to a reporter molecule or a nanoparticle.

In certain aspects, the reporter molecule comprises a hydrophobic reporter molecule. In particular aspects, the hydrophobic reporter molecule comprises a hydrophobic fluorescent maleimide. In more particular aspects, the reporter molecule comprises 4,4-difluoro-4-bora-3a,4a-diaza-s-indacene (BODIPY) or an analog or derivative thereof.

In certain aspects, the nanoparticle comprises one or more poly(beta-amino) esters (PBAEs). In particular aspects, the PBAE comprises a branched PBAE. In more particular aspects, the branched PBAE comprises a carboxylated PBAE (cPBAE). In yet more particular aspects, the branched PBAE comprises a compound of formula (I) or formula (II):

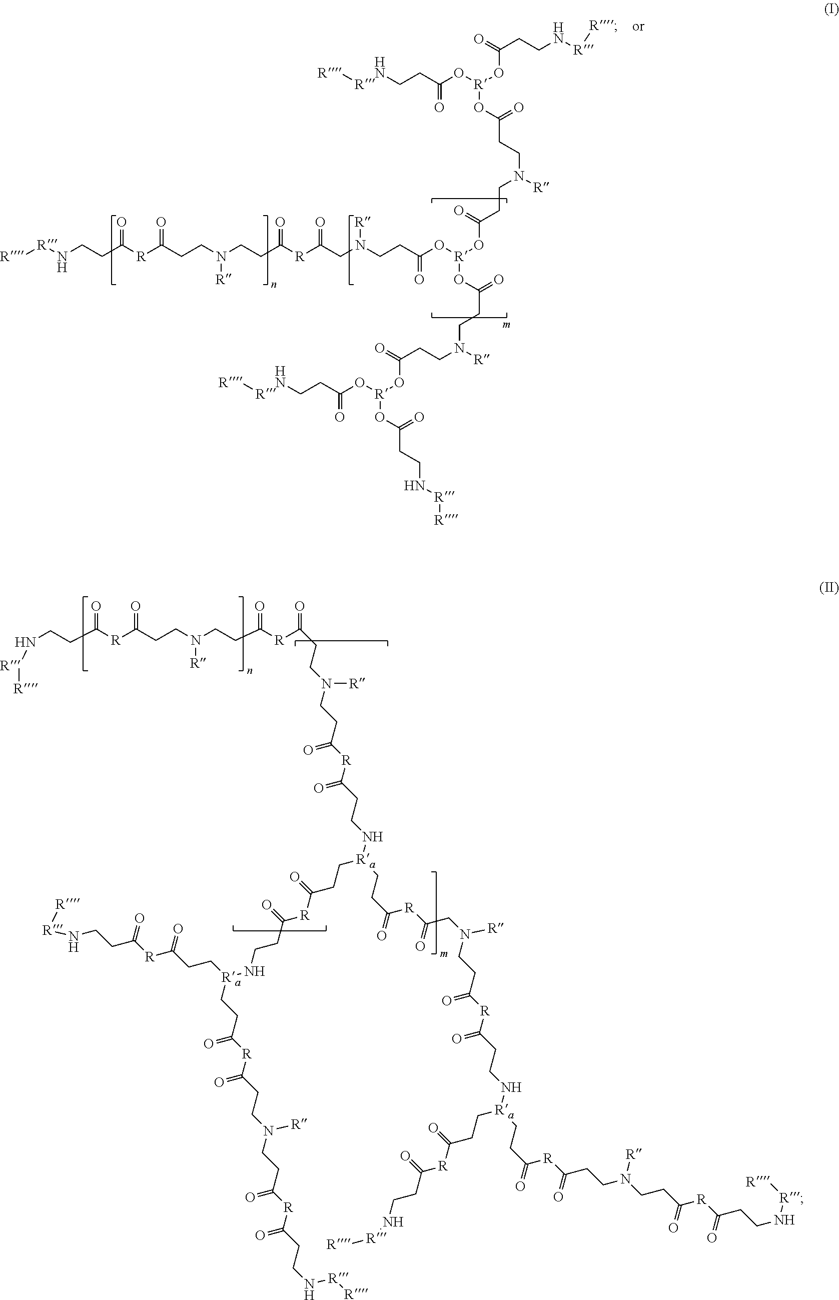

wherein: n and m are each independently an integer from 1 to 10,000; each R is independently a diacrylate monomer of the following structure:

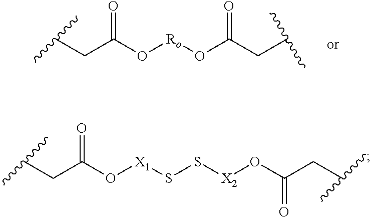

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain; each R′ of formula (I) is a triacrylate, quanternary, or hexafunctional acrylate monomer selected from the group consisting of:

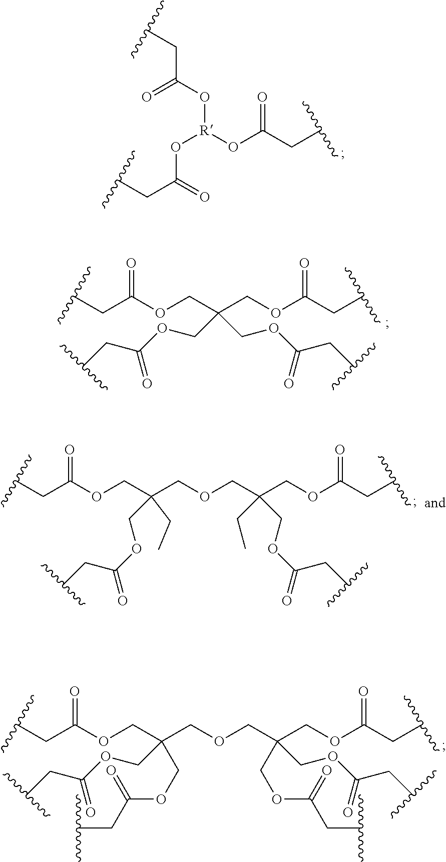

wherein each R′ is independently a trivalent group; each R″ is independently a side chain monomer comprising a primary, secondary, or tertiary amine; and each R″ is independently an end group monomer comprising a primary, secondary, or tertiary amine.

In certain aspects, the nanoparticle and the peptide have a weight-to-weight ratio of about 30:1 nanoparticle: peptide.

In other aspects, the presently disclosed subject matter provides a method for treating a diarrheal disease, the method comprising administering to a subject in need of treatment thereof a therapeutically effective amount of the presently disclosed conjugate.

In certain aspects, the diarrheal disease comprises a diarrheal disease in which NH3 is inhibited. In other aspects, the diarrheal disease comprises a diarrheal disease in which NH3 is not inhibited. In particular aspects, the diarrheal disease is selected from an acute, a persistent, and a chronic diarrheal disease.

In certain aspects, administering a therapeutically effective amount of the peptide treats one or more symptoms of the diarrheal disease. In particular aspects, administering a therapeutically effective amount of the peptide shortens a duration of the diarrheal disease, lessens a volume of diarrhea, and combinations thereof. In more particular aspects, administering a therapeutically effective amount of the peptide increases intestinal Na⁺ absorption, rehydrates the subject, and combinations thereof. In yet more particular aspects, administering a therapeutically effective amount of the peptide stimulates sodium-hydrogen exchanger 3 (NHE3) activity, prevents or diminishes CAMP inhibition of NHE3 activity, reverses reduced NHE3 activity caused by elevated cAMP, cGMP, and Ca²⁺, and inflammatory causes of diarrhea, and combinations thereof.

In certain aspects, administering a therapeutically effective amount of the peptide stimulates sodium-hydrogen exchanger 3 (NHE3) activity, prevents or diminishes CAMP inhibition of NHE3 activity, reverses reduced NHE3 activity caused by elevated cAMP, cGMP, and Ca²⁺, and inflammatory causes of diarrhea, and combinations thereof, in a small intestinal loop of the subject. In particular aspects, the small intestinal loop of the subject includes a jejunum and/or an ileum of the subject.

In certain aspects, the conjugate is delivered to a gastrointestinal tract (GI) tract of the subject. In particular aspects, the conjugate is deposited subapically in the GI tract.

In certain aspects, the subject is an infant.

In certain aspects, the diarrheal disease is selected from watery, fatty (malabsorption), and inflammatory diarrhea. In particular aspects, the diarrheal disease is selected from traveler's diarrhea or diarrhea related to cholera.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Drawings, which are not necessarily drawn to scale, and wherein:

FIG. 1A and FIG. 1B demonstrate that N3SP is part of the NHE3 C-terminus, which forms the inhibitory regulatory complex (IRCX). (FIG. 1A) Domain structure of NHE3 modified from Donowitz et al., 2009 and Hendus-Altenburger et al., 2014, showing N-terminal transport domain and C-terminal regulatory domain including suggested boundaries for the binding of the IRCX. Boxes indicate proteins shown to directly bind to NHE3 C-terminus in the area of the stimulatory and inhibitory regulatory complexes. AA refer to rabbit NHE3. (FIG. 1B) Peptides mimicking the NHE3 amino acids that make up IRCX binding domain (AA refer to rabbit NHE3). CP, control peptide; N3SP, NHE3 stimulatory peptide;

FIG. 2A, FIG. 2B, FIG. 2C, FIG. 2D, and FIG. 2E demonstrate the effect of N3SP-1 on basal and acutely stimulated and inhibited NHE3 activity. (FIG. 2A) Confocal microscopy showing uptake of BODIPY-conjugated N3SP-1 and CP-2 in PS120/HA3-NHE3/NHERF2 cells (top), Caco-2/BBe cells (middle), and mouse jejunum (bottom; two-photon microscopy). BODIPY (left) is not conjugated to either peptide and is not fluorescent (Zachos et al., 2009; Doehning et al., 2005). Red=BODIPY-conjugated peptides. Blue=nuclei. Scale bars=20 μm. (FIG. 2B) Effect of N3SP-1 compared to CP-1 and to BODIPY alone (all studied at 400 nM) on basal and 4-Br-A23187 inhibition of NHE3 activity in PS120/HA3-NHE3/NHERF2 cells. Results are rates of NHE3 activity (μM/sec), mean±SEM of 3 (paired t tests) (FIG. 2C) NHE3 activity in polarized Caco-2/BBe/HA3-NHE3 cells exposed to either 400 nM CP-1 or N3SP-1, both studied under basal conditions, after 5 min exposure to 10-μM carbachol (CCH), 30 min exposure to 25-μM forskolin (FSK), or 30 min exposure to 200-ng/mL epidermal growth factor (EGF). n=3 for all experiments. (paired t tests for comparison with cells treated only with CP-1 or N3SP-1). (FIG. 2D) NHE3 measured in mouse jejunum in vitro studied with two photon microscope/SNARF-4F under basal conditions and after 30 min exposure to 25-μM FSK with exposure to 400-nM CP-1 or N3SP-1. n=6. (unpaired t tests). (FIG. 2E) Kinetic analysis of N3SP-1 stimulation of basal NHE3 activity (blue; EC50=152 nM) and prevention of FSK inhibition of NHE3 (red; EC₅₀=157 nM). EC₅₀ calculated by Hill equation;

FIG. 3A, FIG. 3B, FIG. 3C, FIG. 3D, and FIG. 3E demonstrate that N3SP-1 prevents net mouse jejunal fluid secretion induced by cholera toxin (CTx), E. coli heat stable enterotoxin, and anti-CD3 monoclonal antibody-induced inflammation. (FIG. 3A) Fluid accumulation (loop weight/length) 6 h after inoculation of jejunal loops containing 1-μg purified CTx in presence of 400-nM CP-1, N3SP-1, or PBS p values are comparison with loops inoculated with only PBS (paired t tests). Results from at least 2 loops from 6 animals/condition. Representative loop distention shown below. (FIG. 3B) Intracellular localization of N3SP-1 in mouse jejunum 6 hr after loop inoculation with 1-μg CTx with 400-nM CP-1 or N3SP-1 (upper) and localization of NHE3 under the same conditions (below). Scale bars=20 μm. (FIG. 3C) Fluid accumulation (loop weight/length) 4 h after inoculation of 3-4 cm jejunal loops with 0.5-μg purified E. coli heat stable enterotoxin in presence of 400-nM CP-1, N3SP-1 or PBS. p values are comparison with loops inoculated with only PBS (paired t tests). Results from at least 2 loops from 6 animals/condition. Representative loop distention is shown below. (FIG. 3D) Fluid accumulation (loop wet weight/length) 2.5 h after IP injection of 200-μL PBS or anti-CD3 antibody in mice with two jejunal loops containing 400-nM CP-1 or N3SP-1. p values are comparison of loops inoculated with CP-1 compared to N3SP-1 and loops from IP PBS vs CD3 antibody (paired t tests). Results from 6 animals injected with PBS and 6 with anti-CD3 antibody (total 12 loops studied containing CP-1 or N3SP-1). (FIG. 3E) Studies performed as in FIG. 3A with CTx exposure but with 400-nM CP-2 and N3SP-1Δ7. n=3. p values are comparison with PBS inoculated loops (paired t tests);

FIG. 4A, FIG. 4B, and FIG. 4C demonstrate that nanoparticles-CP-2 and N3SP-1Δ7 are taken up by differentiated human duodenal enteroids, stimulate basal NHE3 activity, and prevent cAMP inhibition of NHE3 activity. (FIG. 4A) Confocal microscopy of fluorescently labeled nanoparticles (PBAECR5 30:1 w/w nanoparticles: peptide) (2 μM, 4 h uptake after apical addition to differentiated enteroid monolayers) revealed predominantly subapical location of nanoparticles (green) (XY sections above; XZ sections below). NP were labeled with streptavidin-Alexa488 conjugated peptides. Endogenous NHE3 (red; anti-NHE3-Alexa567) marked the apical domain. Note lack of uptake of N3SP-1Δ7 in the absence of conjugation to nanoparticles (left panel). Scale bar=10 μm. (FIG. 4B) Differentiated enteroid monolayers were exposed to nanoparticle-CP-2 or N3SP-1Δ7 (250 nM) overnight and NHE3 activity measured under basal conditions or after FSK exposure. n=4. (paired t tests). (FIG. 4C) Adenylate cyclase-cAMP levels were measured on enteroid monolayers treated as in FIG. 4B, comparing results from 12 monolayers from n=3 experiments, p values from unpaired t tests;

FIG. 5A, FIG. 5B, and FIG. 5C demonstrate that nanoparticle conjugated-N3SP-1Δ7 stimulates NHE3 and reduces cholera toxin-induced fluid secretion in vivo. (FIG. 5A) Closed loop absorption studies of fluid absorption over 30 min in in vivo mouse proximal small intestine following 4 h exposure to nanoparticle-peptide (400 nM) demonstrated that N3SP-1Δ7 compared to CP2 increased fluid absorption. (FIG. 5B) The N3SP-1Δ7 increased baseline fluid absorption was prevented by pretreatment with 10-μM Tenapanor. n=4 and 3 separate experiments for FIG. 5A and FIG. 5B, respectively, with 16 animals studied for FIG. 5A and 12 animals studies for FIG. 5B. (FIG. 5C) 4 h after CTx (0.1 μg) exposure, net fluid secretion was determined in ileal loops also inoculated only with PBS, nanoparticles-CP-2 (4 μM) or nanoparticles-N3SP-1Δ7 (4 μM). n=6. p values compared to PBS/Ctx loops by unpaired t tests. In parallel, ileal loops only exposed to PBS represent an additional control (n=5);

FIG. 6 demonstrates that N3SP-1Δ7 preferentially reduces NHERF2 binding to NHE3 peptides in in vitro overlay assays. Two NHE3 C-terminal peptides containing the N3SP-1 sequence were separated on SDS-Page gradient gels and purified NHERF binding determined. Equal concentrations of NHE3 peptides and NHERF2 were used. Prior to NHERF2 addition, the blots were overlayed with 5× the concentration of N3SP-1Δ7 vs CP2. The test peptide competed with NHERF2 binding significantly more than the control peptide. n=5. p values from unpaired t tests;

FIG. 7A and FIG. FIG. 7B show: (FIG. 7A) Fluorescence emission spectra of BODIPY alone vs BODIPY-conjugated to CP-1 and N3SP-1 showing an increase in fluorescence in the presence of peptide with an emission peak of approximately 617 nm. (FIG. 7B) Failure of CP-1 and N3SP-1 (400 nM) to alter basal NHE3 activity in PS120 cells in the absence of BODIPY conjugation. Exposure to CP-1 and N3SP-1 was performed as in studies shown in FIG. 2C. Neither CP-1 nor N3SP-1 altered basal NHE3 activity compared to otherwise untreated control PS120 cells. n=4;

FIG. 8A and FIG. 8B show structural models of NHE peptides. (FIG. 8A) Structural model of N3SP-1 (residues rabbit NHE3 568-605) and N3SP-1-NA10 (residues 578-605) shown in ribbon diagram representation and colored using a rainbow scheme from N-terminal region (blue) to C-terminal region (red). All structural images were generated using the UCSF Rosetta Chimera package (Rohl et al., 2004). (FIG. 8B) Sequence alignment of human, rabbit, rat and mouse NHE3 in the C-terminal area used to design the N3SP;

FIG. 9A, FIG. 9B, and FIG. 9C demonstrate that: (FIG. 9A) CTx-induced electrogenic anion secretion is not altered by N3SP-1 exposure for 6 hours. Mouse jejunal loops were exposed to 1-μg CTx in the presence of 400-nM CP-1 or N3SP-1 or PBS for 6 h. After animal sacrifice, the full thickness loops were mounted in Ussing chambers, gassed with 5% CO₂/95% O₂ in Ringer's —HCO₃— and after stabilization, basal short-circuit current (Isc in μAMPs/cm²) was determined. Shown are Isc in CTx treated loop with no further addition of secretagogues. p values compare Isc in CTx treated loops also inoculated with PBS, CP-1 (400 nM) or N3SP-1 (400 nM) (paired t tests). n=3. The same tissues were further studied by addition of 10-μM FSK to the serosal surface and peak increase in Isc determined. Then after stabilization of the Isc, 10-μM CCH was added to the same jejunal serosal surface and peak Isc determined. (FIG. 9B) The peak increases in Isc in mouse jejunal loops exposed to cholera toxin (CTx) as in FIG. 9A caused sequentially by basolateral exposure to forskolin (10 μM) and carbachol (10 μM). (FIG. 9C) Results from FIG. 3D showing effect of CP-1 versus N3SP-1 on anti-CD3 monoclonal antibody-induced mouse jejunal fluid secretion separated based on sex. Similar results occurred with studies of both males and females. n=3 for all conditions in both male and female mice. p values are paired t tests; and FIG. 10A, FIG. 10B, FIG. 10C, FIG. 10D, FIG. 10E, FIG. 10F, FIG. 10H, and FIG. 10I demonstrate characterization of cPBAE CR5 polymer and nanoparticles. (FIG. 10A) Synthesis scheme for cPBAE CR5 polymer. (FIG. 10B) 1H-NMR spectrum confirming the chemical structure of cPBAE polymer CR5. Peaks correspond to protons labeled in the chemical structures shown. (FIG. 10C) The CR5 polymer was characterized by gel permeation chromatography (GPC) demonstrating an average molecular weight of 3438 Da and a polydispersity of 1.30. (FIG. 10D and FIG. 10E) Normalized diameter and zeta potential of nanoparticles with CP-2 and N3SP-1Δ7. To form nanoparticles, CR5 was combined with CP-2/N3SP-1Δ7 in aqueous conditions at a 30:1 polymer: peptide weight ratio. The resulting self-assembled nanoparticles had a diameter of 319±55 nm (CP-2) and 385±30 nm (N3SP-1Δ7), as measured by dynamic light scattering (DLS), with a surface zeta potential of ±24.6±0.5 mV (CP-2) and ±22.4±1.2 mV (N3SP-1Δ7) measured by electrophoretic light scattering. Nanoparticles incubated in PBS for 45 minutes at room temperature remained stable, with no significant increase in size. (FIG. 10F) Morphology of PBAE-peptide (N3SP-1Δ7 shown) complexes by transmission electron microscopy. Scale bars represent 100 nm. The expected loosely formed complexes are demonstrated. (FIG. 10G.) Effect of pH (pH 1.2, 4.0, 7.0) on nanoparticle-peptide compared to peptide alone on viability and uptake in B16F10 cells. Left: % viability; Middle: % uptake; and Right: geometric mean intensity (size). n=4. Significance determined in Graphpad Prism by one way ANOVA with Tukey's multiple correction test. ns=p>0.05, *=p<0.05, **=p<0.01, *=p<0.001, ****=p<0.0001. (FIG. 10H) Effects of pH (pH 1.2, 4.0, 7.0) on nanoparticle diameter by dynamic light scattering (DLS). Left: average particle diameter measured by DLS after nanoparticle-peptide or peptide alone exposure to pH 1.2, 4.0, 7.0; Middle: particle polydispersity after exposure to varying pH conditions; Right: peak one mean measured via DLS of particles exposed to varying pH conditions. n=6. Significance determined in Graphpad Prism by one way ANOVA with Tukey's multiple correction test. ns=p>0.05, *=p<0.05, **=p<0.01, ***=p<0.001, ****=p<0.0001. (FIG. 10I) Time and concentration dependence of nanoparticle-peptide uptake in vitro in B16-F10 cancer cells (% positive). When nanoparticles were added to B16-F10 cells in vitro at concentrations from 0.25 μM to 2 μM, dose-dependent cellular uptake was observed, with significant, time dependent uptake over 4 h. Time and concentration dependent uptake of nanoparticles formulated with fluorescently labeled CP-1 (left) and N3SP-1 (right) (% positive shown).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter, in part, provides a 31 amino acid (aa) peptide that is part of the epithelial sodium-hydrogen exchanger 3 (NHE3). In some embodiments, the peptide is linked to a nanoparticle for delivery to the gastrointestinal tract (GI) tract. NHE3 is the major way in which sodium (Na⁺) is absorbed in the intestine between meals and in the kidney. NHE3 is regulated acutely by complexes of proteins that stimulate and inhibit NHE3. The peptide mimics the place the inhibitory complex binds and removes the inhibitory complex resulting in stimulation of NHE3. The nanoparticle allows delivery into the intestine and is pH stable. The peptide stimulates intestinal sodium absorption and diarrhea in mouse models of cholera, traveler's diarrhea, and inflammation-related diarrhea. The peptide, however, is not taken up by the intestine when exposed apically unless it is attached to a material to allow for uptake: this is the role of the nanopeptide.

### A. NHE3 Mimetic Peptides

In some embodiments, the presently disclosed subject matter provides an NHE3 mimetic peptide. More particularly, a series of peptides were synthesized to mimic the region of the NHE3 C-terminus that forms a multi-protein complex that inhibits NHE3 activity. Thesc peptides were based on the aa 586-605 region (rabbit NHE3) of the intracellular C-terminal domain, termed the NHE3 Inhibitory Regulatory Complex (IRCX), which directly binds proteins that inhibit NHE3 activity.

Initially, a 21 aa peptide representing aa 585-605 of rabbit NHE3 was synthesized with an additional C-terminal Cys to conjugate BODIPY 577/618, a fluorescent maleimide that when conjugated to a peptide/protein renders the peptide cell-permeable and fluorescent. There was no significant effect, however, of this peptide, referred to as CP-1, compared to BODIPY alone on basal NHE3 activity.

A bioinformatics approach was used to provide insight into aa immediately upstream or downstream from the CP-1 boundaries. Extending the N-terminal sequence of CP-1 to aa 568, while keeping the C-terminal aa 605 provided a peptide named N3SP-1. Removing up to 7 amino acids from the N-terminus of this peptide preserved the ability to stimulate NHE3 activity. Truncation of 8 aa, however, reduced the stimulatory effect, while further truncations of 9, 10 and 18 aa produced peptides that had no effect on basal NHE3 activity.

Accordingly, in some embodiments, the presently disclosed subject matter provides an NHE3 mimetic peptide selected from NHE3 stimulatory peptide-1 (N3SP-1), N3SP-1Δ7, N3SP-1A6, and N3SP-1Δ5. In some embodiments, the peptide is N3SP-1. In some embodiments, the peptide is N3SP-1Δ7. In some embodiments, the peptide is N3SP-1Δ6. In some embodiments, the peptide is N3SP-1Δ5. In particular embodiments, the NHE3 mimetic peptide is N3SP-1Δ7. Sequences of these peptides are provided in Table 1.

TABLE 1
Representative NHE3 Mimetic Peptides.

| Peptide | SEQ ID NO: | Amino Acid Sequence |
|---|---|---|
| N3SP- | SEQ ID | NVDFSTPRPSTVEASVSYLLRESASAVCLDM |
| 1 | NO: 1 | QSLEQRR |
| N3SP- | SEQ ID | RPSTVEASVSYLLRESASAVCLDMQSLEQRR |
| 1Δ7 | NO: 2 | |
| N3SP- | SEQ ID | PRPSTVEASVSYLLRESASAVCLDMQSLEQR |
| 1Δ6 | NO:3 | R |
| N3SP- | SEQ ID | TPRPSTVEASVSYLLRESASAVCLDMQSLEQ |
| 1Δ5 | NO: 4 | RR |

Note that the sequences provided in Table 1 can include an additional C-terminal Cys for conjugating to a ligand, e.g., a reporter molecule, such as a fluorescent dyc. For conjugation to nanoparticles, an N-terminal biotin can be added to the peptide.

### B. Peptide Conjugates

In some embodiments, the peptide is conjugated to a ligand. The ligand renders the peptide cell-permeable and detectable within the cell, e.g., by fluorescence. In particular embodiments, the ligand comprises a fluorescent label. In more particular embodiments, the fluorescent label comprises a BODIPY dye (4,4-difluoro-1,3,5,7,8-pentamethyl-4-bora-3a,4a-diaza-s-indacene), or an analog or derivative thereof, including, but not limited to, BODIPY 577/618, BODIPY 630/650, BODIPY 650/665, BODIPY 581/591, BODIPY-FL, BODIPY-R6G, BODIPY-TR, BODIPY-TMR, BODIPY-TRX. In yet more particular embodiments, the peptide is conjugated to a BODIPY maleimide.

One of ordinary skill in the art would recognize that other fluorescent dyes are suitable for use with the presently disclosed subject matter including, but not limited to, fluorescein isothiocyanate (FITC), green fluorescent protein (GFP), AlexaFluor 350, AlexaFluor 430, AlexaFluor405, AlexaFluor488, AlexaFluor546, AlexaFluor555, AlexaFluor594, AlexaFluor660, AlexaFluor633, AlexaFluor647, AlexaFluor680, AlexaFluor700, AlexaFluor750, AlexaFluor790, AMCA, Cascade Blue, Cy3, Cy5, Cy5.5, Cy7, 6-FAM, fluorescein, Fluorescein Isothiocyanate, TRITC, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, Texas Red, carbocyanine, indocarbocyanine, oxacarbocyanine, thuicarbocyanine, merocyanine, polymethine, coumarine, rhodamine, xanthene, a boron-dipyrromethane VivoTag-680, VivoTag-S680, VivoTag-S750, Dy677, Dy676, Dy682, Dy752, Dy780, DyLight547, DyLight647, DyLight 350 (Ex/Em=353 nm/432 nm), DyLight 405 (400/420), DyLight 488 (493/518), DyLight 550 (562/576), DyLight 594 (593/618), DyLight 633 (638/658), DyLight 650 (652/672), DyLight 680 (692/712), DyLight 755 (754/776), DyLight 800 (777/794), and derivatives thereof, including, but not limited to, NHS esters, maleimides, phosphines, and free acids, HiLyte Fluor 647, HiLyte Fluor 680, HiLyte Fluor 750, IR800 (EHmethyl {4-[1,5,5-tris(4-dimethylaminophenyl)-2,4-pentadienylidene]-2,5-cyclohexadien-1-ylidene}ammonium perchlorate), IRDye 800CW, IRDye 800RS, IRDye 700DX, ADS780WS, ADS830WS, ADS832WS, R-Phycoerythrin, Flamma749, Flamma774, and ICG.

C. Carboxylated Branched Poly(β-amino Ester) Peptide Nanoparticles

In some embodiments, the presently disclosed subject matter provides compositions, including particles, such as nanoparticles, comprising carboxylated, branched poly(β-amino ester) (PBAE) polymers for delivery of peptides to cells. Such PBAEs have a backbone constructed from diacrylate or other monomers in combination with triacrylate monomers to provide polymers with variable branching. The polymers can be prepared by condensing side chain monomers comprising secondary amines or primary amines with acrylate ester monomers, e.g., diacrylate and triacrylate monomers. For example, in some embodiments, the PBAE comprises a backbone of a diacrylate, e.g., bisphenol A glycerolate (1 glycerol/phenol) diacrylate (BGDA), a backbone comprising a disulfide (e.g., BR6), and a triacrylate, e.g., trimethylolpropane triacrylate (TMPTA).

In some embodiments, the polymers comprise tertiary amines in their backbone and/or in some embodiments, the polymers comprise side chains and/or end groups comprising primary, secondary, and/or tertiary amines. In some embodiments, the secondary or tertiary amines comprise bivalent amine-containing heterocyclic groups. In some embodiments, the side chain monomers comprise a primary amine, but also may comprise secondary and tertiary amines. In some embodiments, the end group terminates with a primary amine and a hydroxyl, with an internally placed secondary amine.

The presently disclosed polymers have the property of biphasic degradation and modifications to the polymer structure can result in a change in the release of therapeutic agents, e.g., a peptide. In some embodiments, the presently disclosed polymers include a minority structure, e.g., an end-capping group, which differs from the majority structure comprising most of the polymer backbone. In other embodiments, the bioreducible oligomers form block copolymers with hydrolytically degradable oligomers. In yet other embodiments, the end group/minority structure comprises an amino acid or chain of amino acids, or, in particular embodiments, carboxylate ligands synthesized via acrylation of amino acid derivatives to yield a series of acrylated amino acids with varying numbers of carbon atoms between the carboxyl and amide groups, while the backbone degrades hydrolytically and/or is bioreducible.

Small changes in the monomer ratio used during polymerization, in combination with modifications to the chemical structure of the end-capping groups used post-polymerization, can affect the efficacy of delivery of a peptide to a cell. Further, changes in the chemical structure of the polymer, either in the backbone of the polymer or end-capping groups, or both, can change the efficacy of peptide delivery to a cell. In some embodiments, small changes to the molecular weight of the polymer or changes to the end-capping groups of the polymer, while leaving the main chain, i.e., backbone, of the polymer the same, can enhance or decrease the overall delivery of the peptide to a cell. Further, the monomer groups that comprise the backbone or main chain of the polymer can be selected to degrade via different biodegradation mechanisms within the same polymer molecule. Such mechanisms include, but are not limited to, hydrolytic, bioreducible, enzymatic, and/or other modes of degradation.

The properties of the presently disclosed carboxylated, branched PBAEs can be tuned to impart one or more of the following characteristics to the composition: independent control of cell-specific uptake and/or intracellular delivery of a particle; independent control of endosomal buffering and endosomal escape; independent control of peptide release; triggered release of an active agent; modification of a particle surface charge; increased diffusion through a cytoplasm of a cell; increased active transport through a cytoplasm of a cell; increased nuclear import within a cell; and/or increased persistence of an associated therapeutic agent within a cell.

If a hydrophilic peptide is to be encapsulated, a hydrophilic polymer is chosen as the multicomponent material. If a hydrophobic peptide is to be encapsulated than a hydrophobic polymer is chosen. The polymer backbone, side chain, and/or terminal group can be modified to increase the hydrophobic or hydrophilic character of the polymer. The peptide to be encapsulated can be first dissolved in a suitable solvent, such as DMSO or PBS. Then, it is combined with the polymer in, for example, sodium acetate (NaAc). This solution is then diluted with either sodium acetate, OptiMem, DMEM, PBS, or water depending on the particle size desired. The solution in vortexed to mix and then left to incubate for a period of time for particle assembly to take place. The particles can self-assemble with a peptide to form nanoparticles that can be in the range of 50 nm to 500 nm in size.

Representative multicomponent degradable cationic polymers are disclosed in the following U.S. patents and U.S. patent application publications, each of which is incorporated herein by reference in its entirety:

U.S. Patent Application Publication No. 20180177881 for Multicomponent Degradable Cationic Polymers, to Green et al., published Jun. 28, 2018;

U.S. Patent Application Publication No. 20150250881 for Multicomponent Degradable Cationic Polymers, to Green et al., published Sep. 10, 2015;

U.S. Patent Application Publication No. 20120128782 for Multicomponent Degradable Cationic Polymers, to Green et al., published May 24, 2012;

U.S. Patent Application Publication No. 20180112038 for Poly(beta-amino ester)-co-polyethylene glycol (PEG-PBAE-PEG) Polymers for Gene and Drug Delivery, to Green et al., published Apr. 26, 2018;

U.S. Patent Application Publication No. 20180028455 for Peptide/Particle Delivery Systems, to Green et al., published Feb. 1, 2018; U.S. Patent Application Publication No. 20160374949 for Peptide/Particle Delivery Systems, to Green et al., published Dec. 29, 2016;

U.S. Patent Application Publication No. 20120114759 for Peptide/Particle Delivery Systems, to Green et al., published Dec. 29, 2016;

U.S. Patent Application Publication No. 20160122390 for A Biomimetic Peptide and Biodegradable Delivery Platform for the Treatment of Angiogenesis- and Lymphangiogenesis-Dependent Diseases, to Popel, et al, published May 5, 2016;

U.S. Patent Application Publication No. 20150273071 for Bioreducible Poly(Beta-Amino Ester) s for siRNA Delivery, to Green et al., published Oct. 1, 2015;

U.S. Pat. No. 9,884,118 for Multicomponent Degradable Cationic Polymers, to Green, et al., issued Feb. 6, 2018;

U.S. Pat. No. 9,717,694 for Peptide/particle Delivery Systems, Green, et al., issued Aug. 1, 2017; and

U.S. Pat. No. 8,992,991 for Multicomponent Degradable Cationic Polymers, to Green, et al., issued Mar. 31, 2015; U.S. Pat. No. 8,287,849 for Biodegradable Poly(beta-amino esters) and Uses Thereof, to Langer, et al., issued Oct. 16, 2012. Other exemplary PBAE polymers are described in WO/2012/0128782, WO/2012/0114759, WO/2014/066811, WO/2014/066898, and US2016/0122390, each of which is incorporated herein by reference in its entirety. In some embodiments, the presently disclosed particles can comprise a polymer blend of PBAEs, e.g., a mixture of PBAE polymers.

Generally, the presently disclosed PBAEs include a backbone derived from a diacrylate monomer (designated herein below as “B”), an amino-alcohol side chain monomer (designated herein below as “S”), and an amine-containing end-cap monomer (designated herein below as “E”). The end group structures are distinct and separate from the polymer backbone structures and the side chain structures of the intermediate precursor molecule for a given polymeric material. The presently disclosed PBAE compositions can be designated, for example, as B5-S4-E7 or 547, in which R is B5, R″ is S4, and R′ is E7, and the like, where B is for backbone and S is for the side chain, followed by the number of carbons in their hydrocarbon chain. End-capping monomers, E, are sequentially numbered according to similarities in their amine structures.

In particular embodiments, the presently disclosed polymers have a backbone constructed from a triacrylate monomer to provide polymers with variable branching.

In particular embodiments, the presently disclosed subject matter provides poly(β-amino esters (PBAE's) exhibiting polymer branching and having carboxylate end-groups for delivery of the presently disclosed peptides to the GI tract of a subject. A representative schematic of the preparation of such PBAE's is provided in FIG. 10A. In the example shown in FIG. 10A, S4 is a titratable tertiary amine side chain monomer; B7 is a backbone polymer having a hydrolysable ester bond; BR6 is a backbone polymer having a bioreducible disulfide linkage; B8 is a branched polymer having a hydrolysable ester bond; E1 is a cationic secondary amine end-capping group; and C5 is a carboxylate ligand. The resulting carboxylated branched PBAE is designated herein as “CR5.”

Representative monomers suitable for use in preparing the presently disclosed PBAE branched polymer are provided hereinbelow. Such monomers can be used to prepare a so-called “base polymer” having a cationic secondary amine end-capping group, i.e., an “E” monomer, such as “E1.” The base polymer can be reacted with a carboxylate ligand to form a carboxylated branched PBAE. Representative carboxylate ligands designated as “C1,” “C3,” “C5,” “C7,” and “C10” are provided immediately herein below:

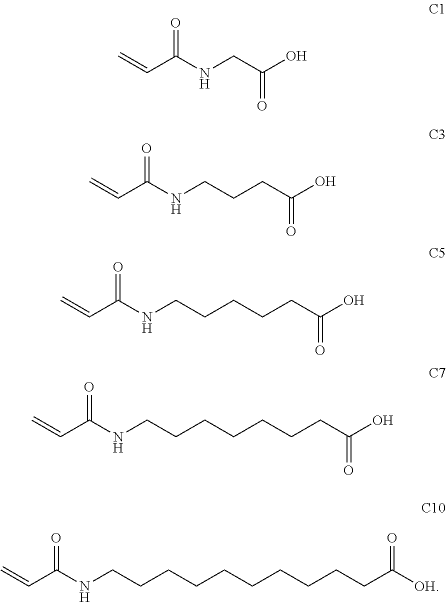

Representative carboxylated branched PBAE polymers suitable for use in the presently disclosed subject matter are described in:

WO2021081495 for Polymeric Nanoparticles for Intracellular Protein Delivery, to Green et al., published Apr. 29, 2021; and

WO2020077159 for Poly(Beta-Amino Ester) Nanoparticles for the Non-Viral Delivery of Plasmid DNA for Gene Editing and Retinal Gene Therapy, to Green et al., published Apr. 16, 2020, each of which is incorporated by reference in its entirety.

More particularly, in some embodiments, the presently disclosed subject matter provides a composition comprising and at least one peptide of SEQ ID NO:1, SEQ ID NO:2, SEQ ID NO:3, or SEQ ID NO:4 conjugated with a branched poly(beta-amino ester) (PBAE) of formula (I) or formula (II):

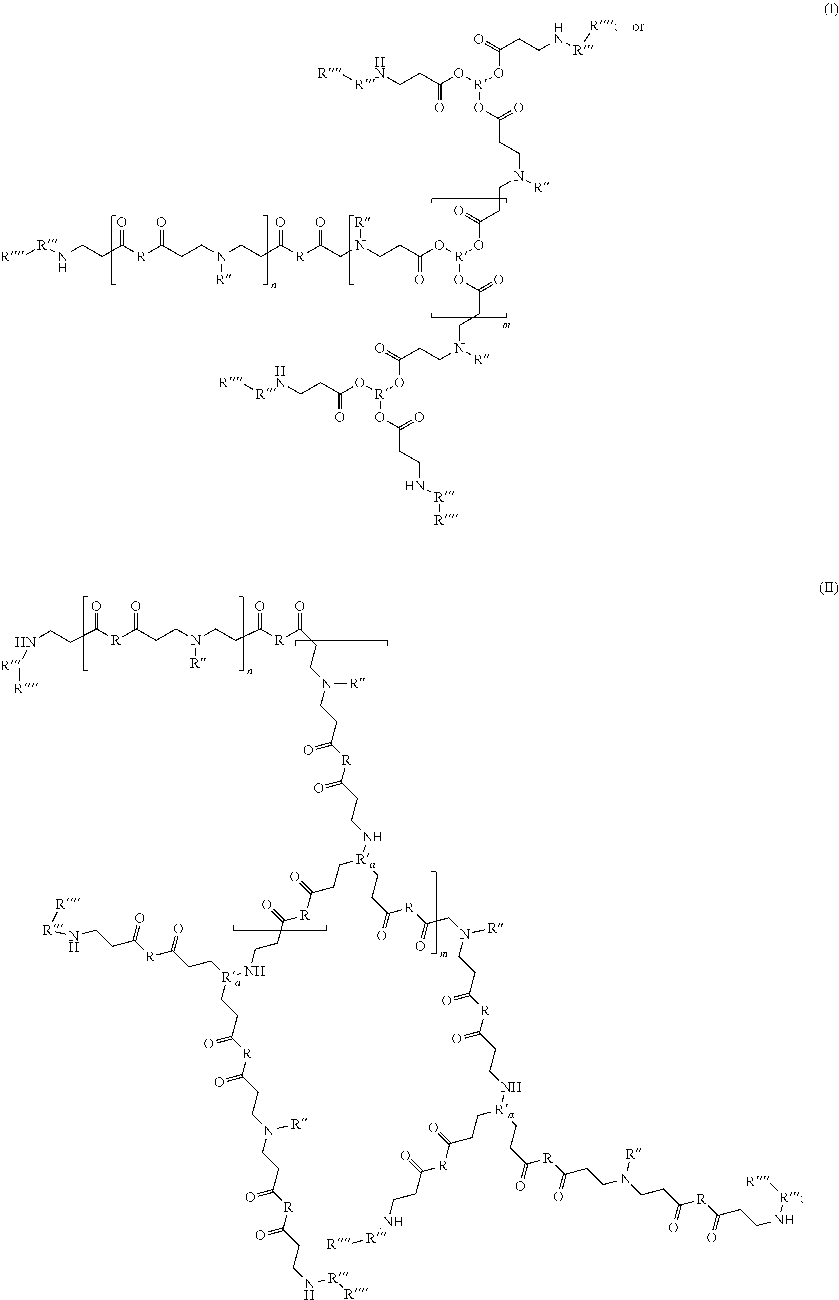

wherein: n and m are each independently an integer from 1 to 10,000; each R is independently a diacrylate monomer of the following structure:

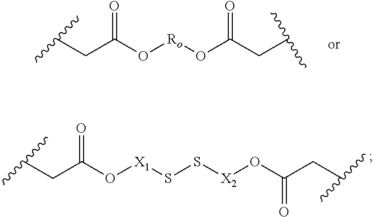

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain; each R′ of formula (I) is a triacrylate, quanternary, or hexafunctional acrylate monomer selected from the group consisting of:

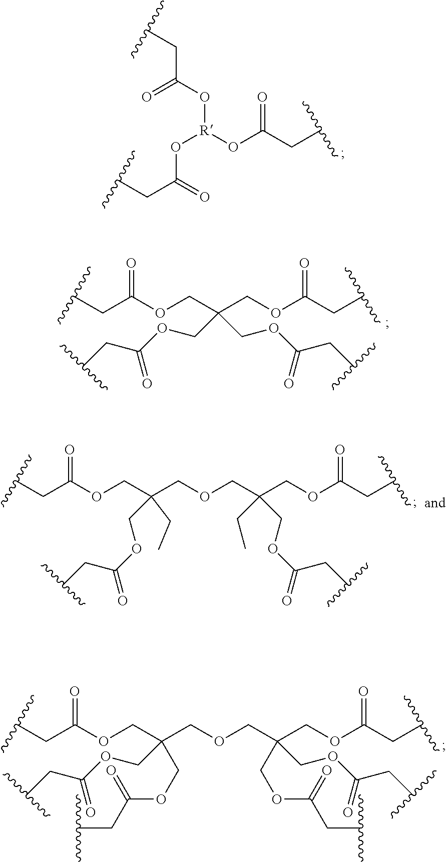

wherein each R′ is independently a trivalent group; each R″ is independently a side chain monomer comprising a primary, secondary, or tertiary amine; and each R″ is independently an end group monomer comprising a primary, secondary, or tertiary amine.

In particular embodiments, R′ is:

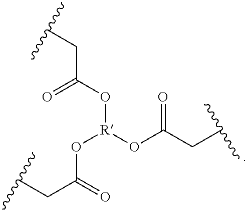

Embodiments comprising the composition of formula (II), which has a tri-functional amine as the linking branching unit, can be prepared as follows:

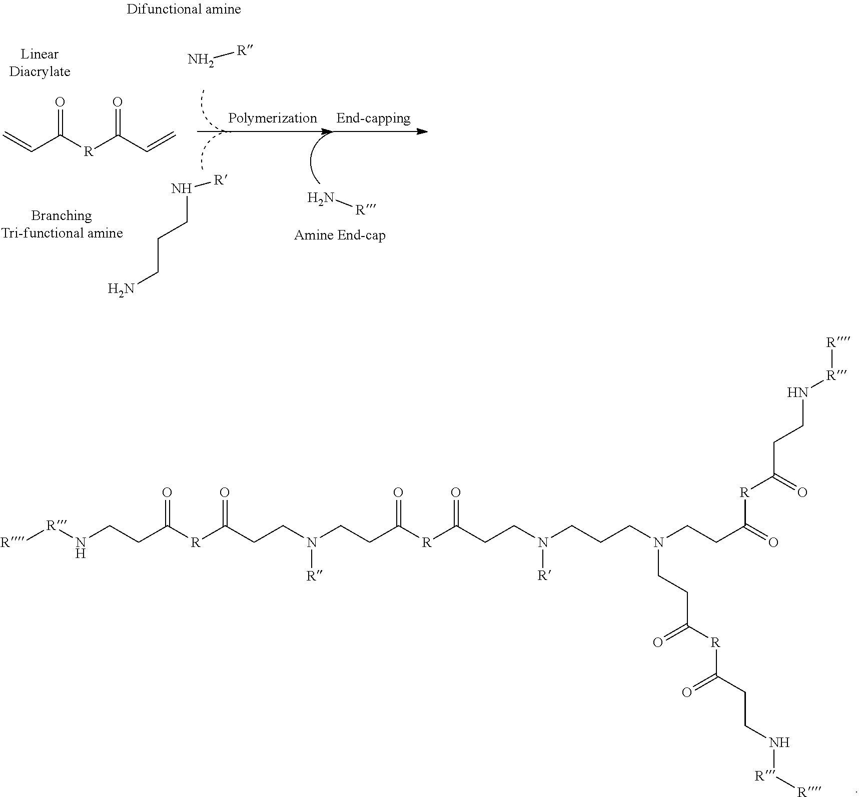

One of ordinary skill in the art would recognize that any amine-containing monomer with either one primary and one secondary or three secondary amines would be suitable for use as the branching tri-functional amine with the presently disclosed compositions of formula (II).

In some embodiments of the composition of formula (I) or formula (II), R is selected from the group consisting of:

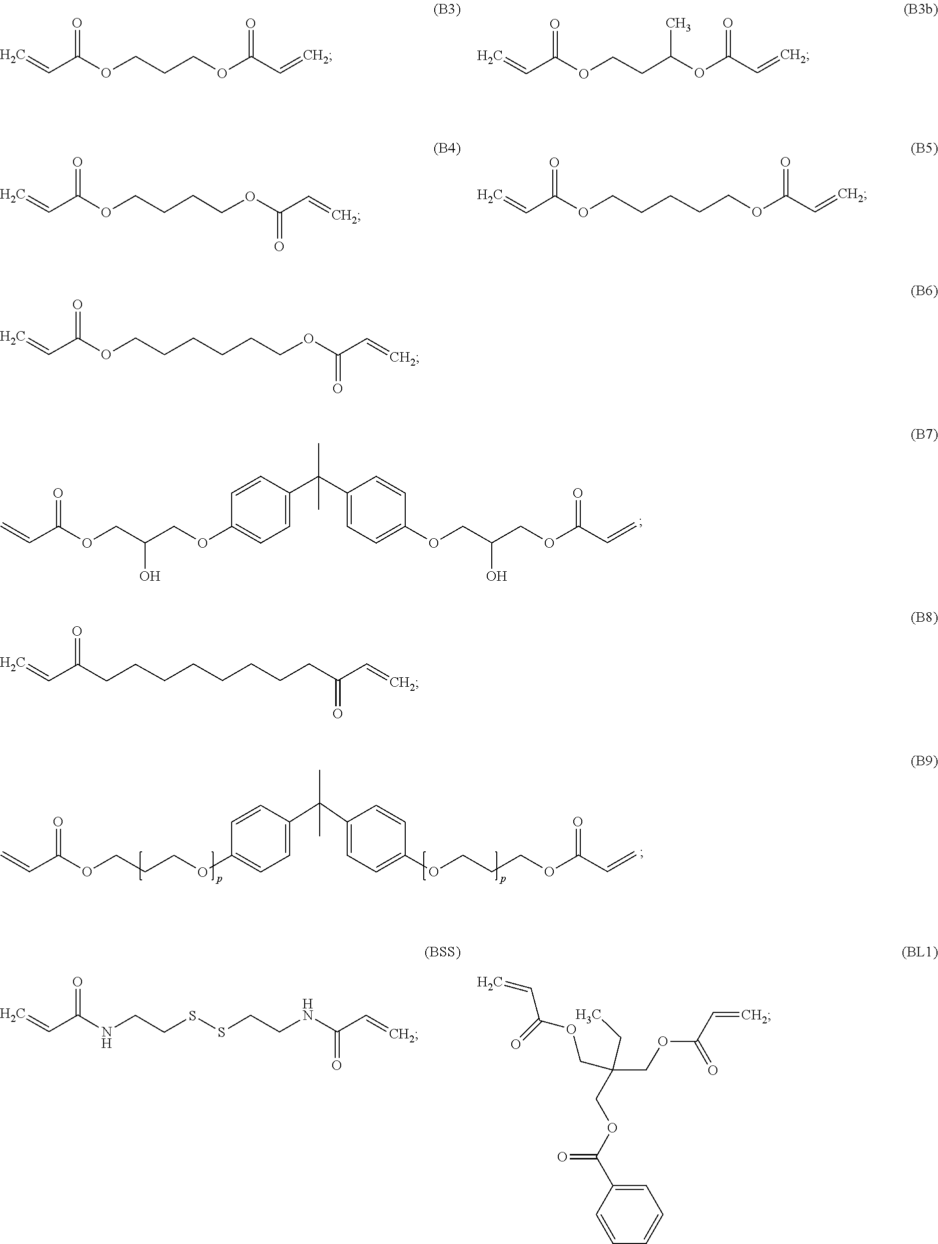

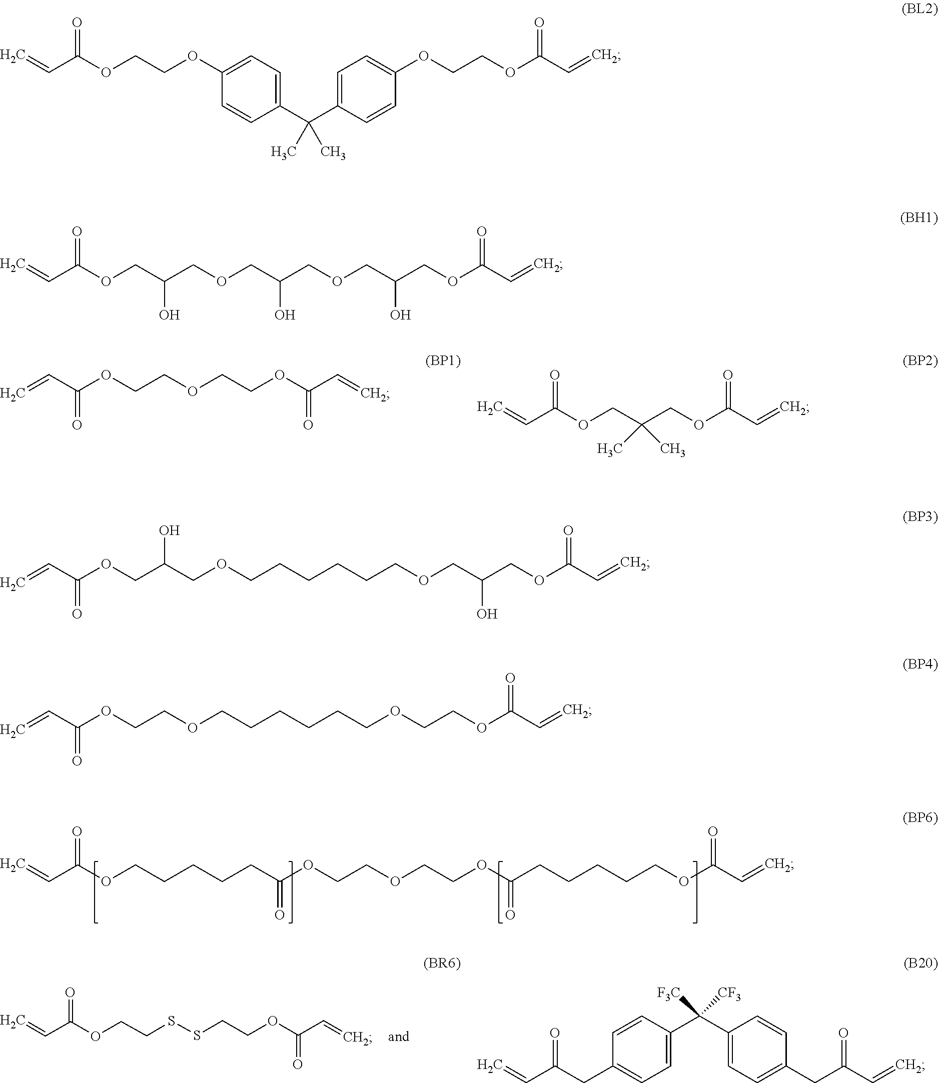

wherein p, q, and u are each independently an integer from 1 to 10,000. In some embodiments of the composition of formula (I), the trivalent group R′ is —C—CH₂CH₃ and the triacrylate monomer is trimethylolpropane triacrylate (TMPTA):

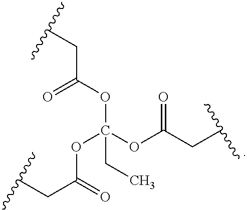 [SMILES: *CC(=O)OC(CC)(OC(=O)C*)OC(=O)C*]

In other embodiments of the composition of formula (II), the tri-functional amine monomer is selected from the group consisting of:

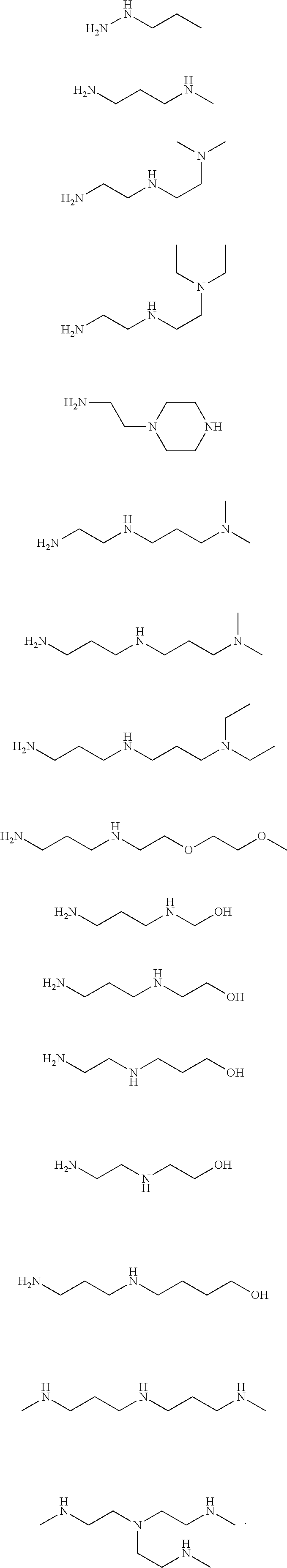

In some embodiments of the composition of formula (I) or formula (II), R″ is selected from the group consisting of:

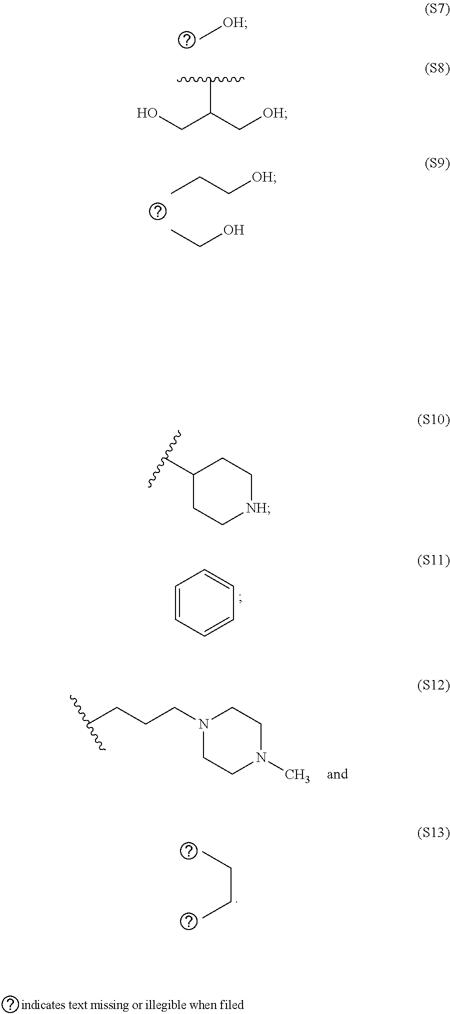

In other embodiments of the composition of formula (I) or formula (II), R″ is selected from the group consisting of:

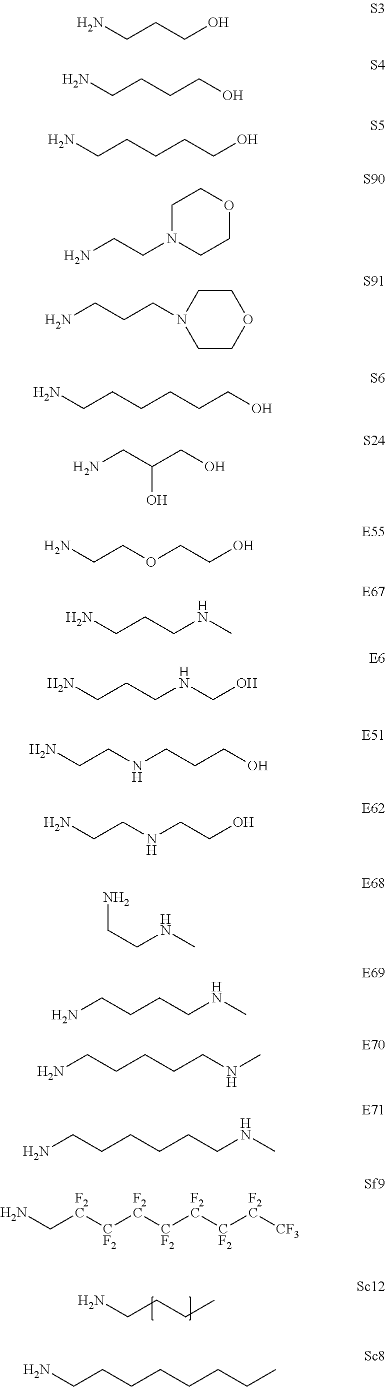

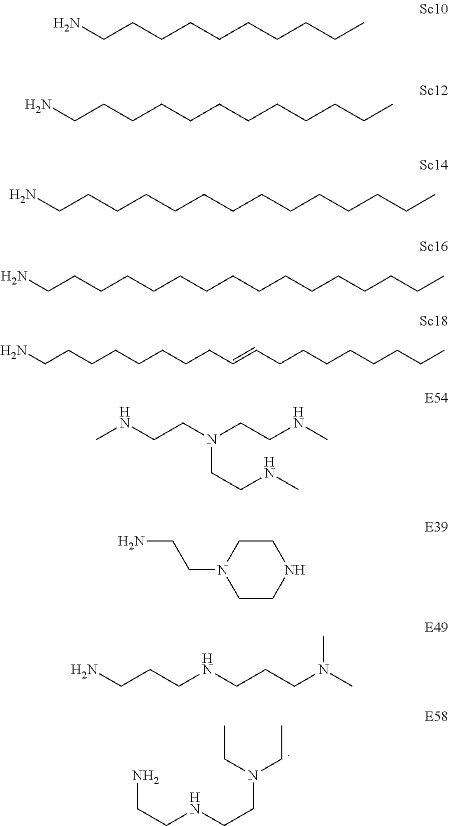

In some embodiments of the composition of formula (I) or formula (II), R″ is selected from the group consisting of:

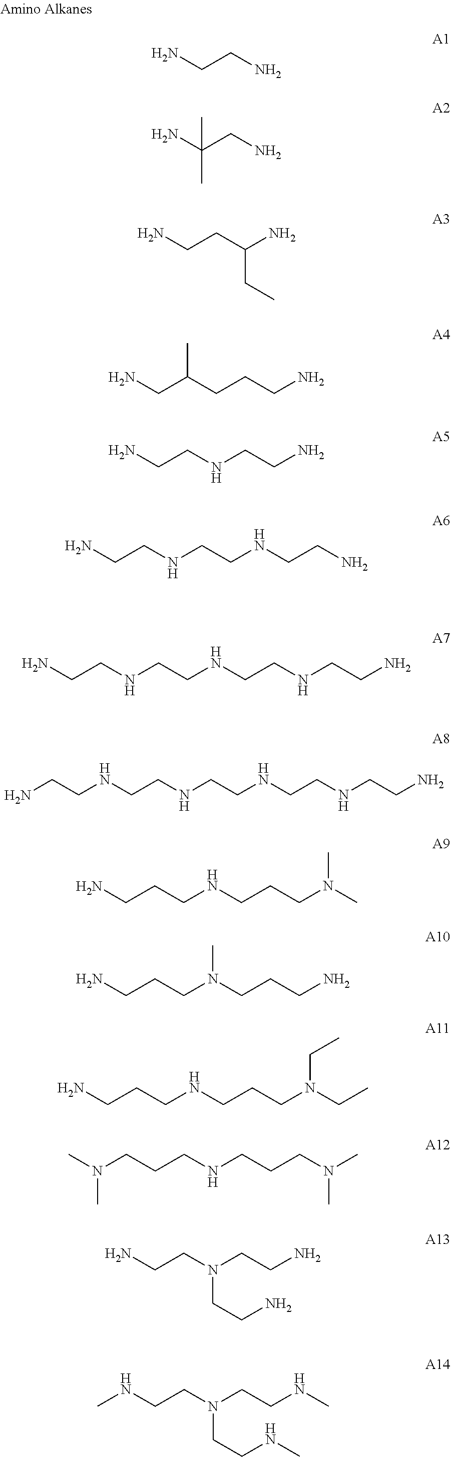

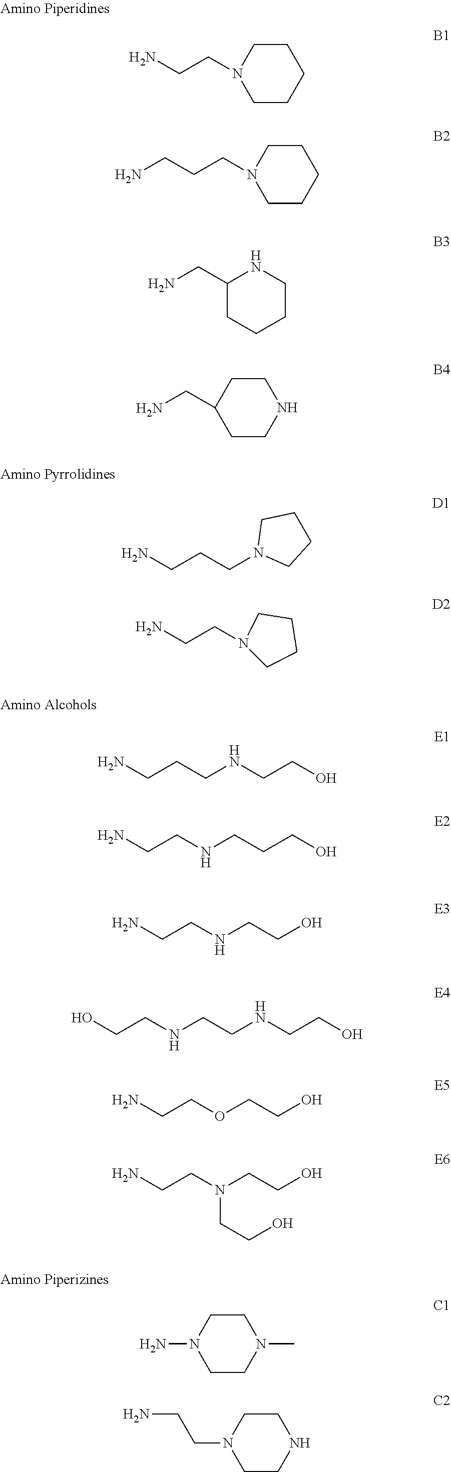

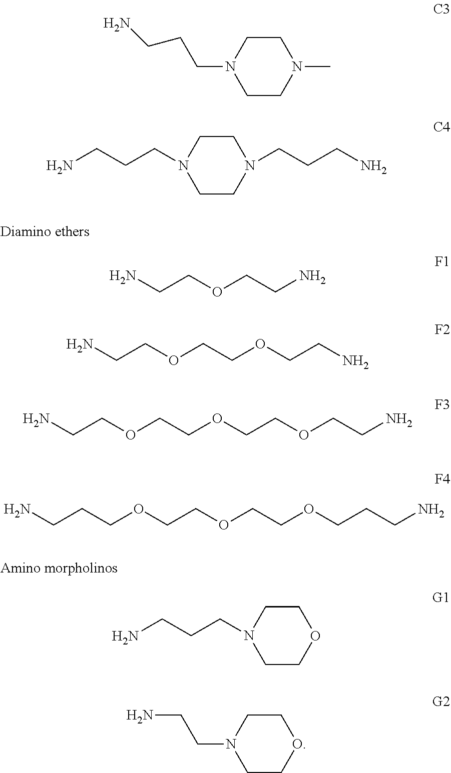

In other embodiments of the composition of formula (I) or formula (II), R′″ is selected from the group consisting of:

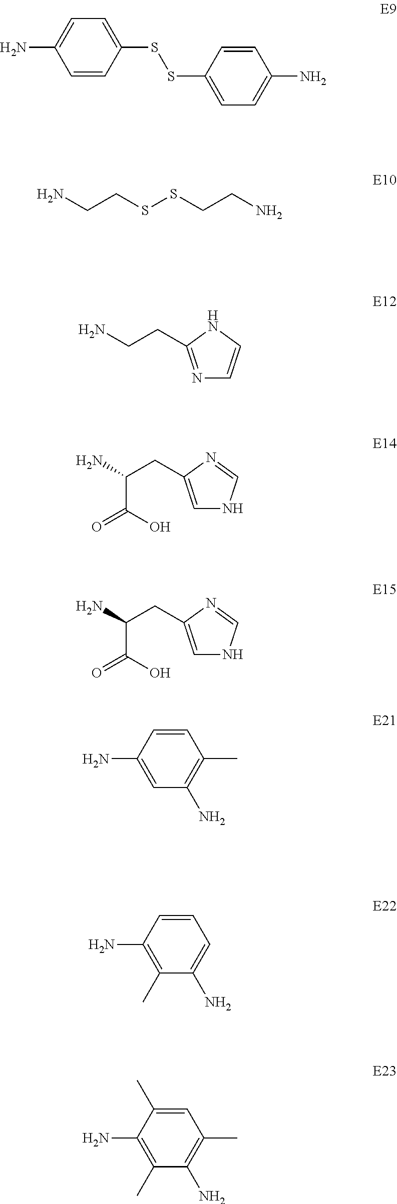

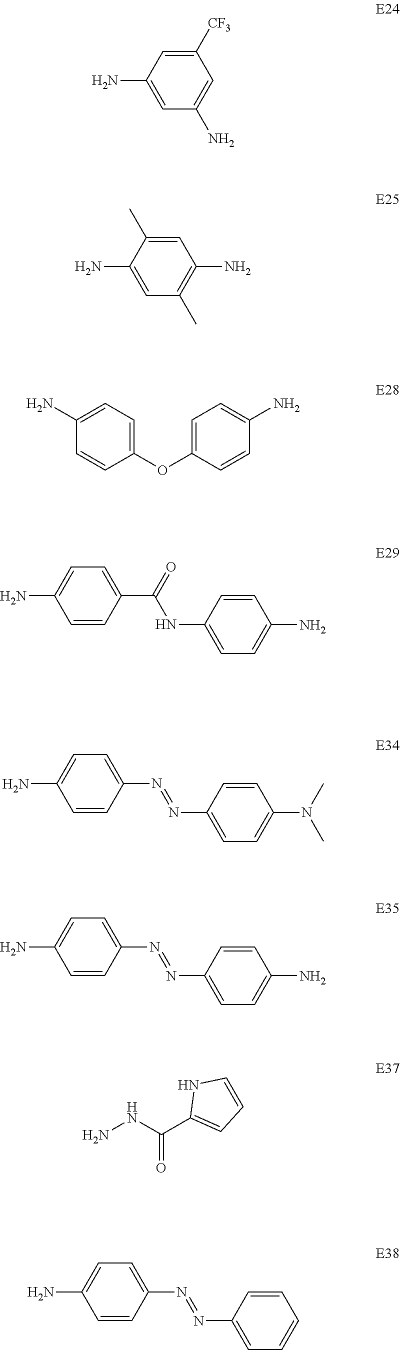

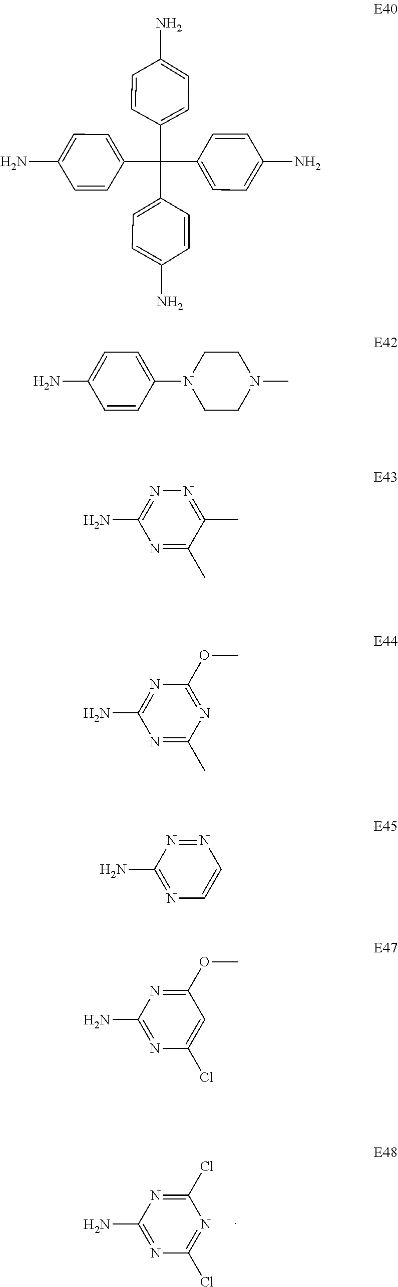

In some embodiments of the composition of formula (I) or formula (II), R″″ is selected from the group consisting of:

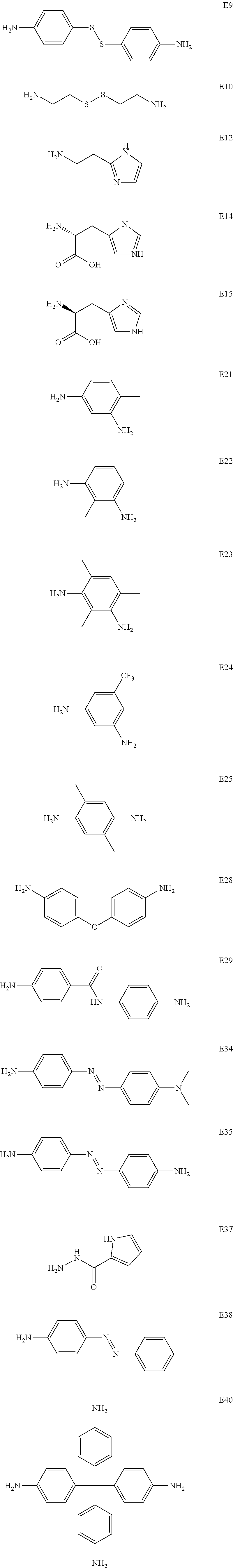

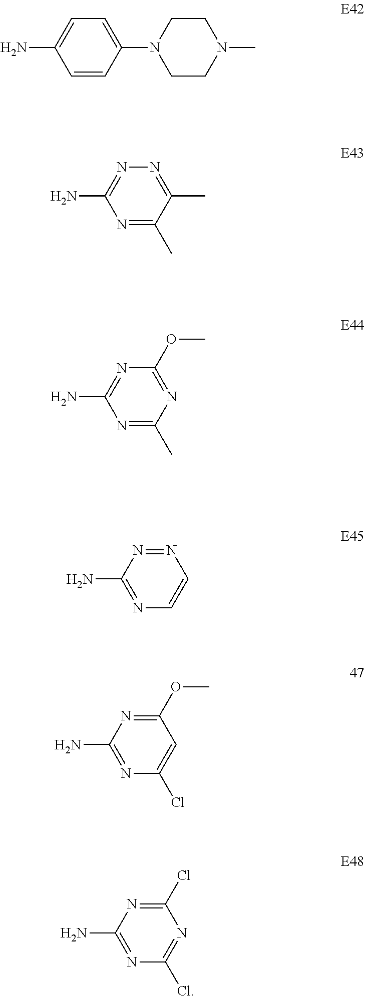

wherein p is an integer from 1, 2, 3, 4, 5, 6, 7, 8, 9, and 10.

In particular embodiments of the composition of formula (I) or formula (II), R″″ is:

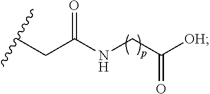

wherein p is an integer from 1, 2, 3, 4, 5, 6, 7, 8, 9, and 10.

In yet more particular embodiments of the composition of formula (I) or formula (II), R″″ is selected from the group consisting of:

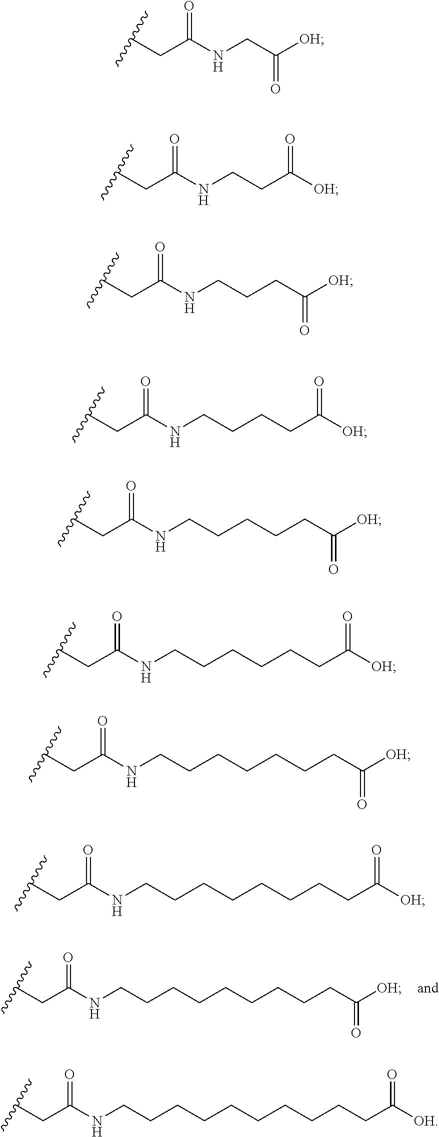

In certain embodiments of a composition of formula (I) or formula (II), n and m are each independently selected from the group consisting of: an integer from 1 to 1,000; an integer from 1 to 100; an integer from 1 to 30; an integer from 5 to 20; an integer from 10 to 15; and an integer from 1 to 10.

In certain embodiments, the branched PBAE polymer has a molecular weight of from 1 to 10 kDa, or a molecular weight of from 10 to 15 kDa, or a molecular weight of from 15 to 25 kDa, or a molecular weight of from 25 to 50 kDa. In particular embodiments, the branched PBAE polymer has an average molecular weight of between about 3,000 Da and about 4,000 Da, including about 3,000 Da, about 3,100 Da, about 3,200 Da, about 3,300 Da, about 3,400 Da, about 3,500 Da, about 3,600 Da, about 3,700 Da, about 3,800 Da, about 3,900 Da, and about 4,000 Da.

In some embodiments, the nanoparticle and the peptide have a weight-to-weight ratio of between about 50:1 to about 5:1 nanoparticle: peptide, including about 50:1, 45:1, 40:1, 35:1, 30:1, 25:1, 20:1, 15:1, 10:1, and 5:1. In particular embodiments, the nanoparticle and the peptide have a weight-to weight ratio of about 30:1 nanoparticle: peptide.

In certain embodiments, the presently disclosed subject matter provides a pharmaceutical formulation of comprising the PBAE composition of formula (I) or formula (II) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

The PBAE polymers in some embodiments can self-assemble with the peptide to form nanoparticles which may be in the range of 50 to 500 nm in size, e.g., about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm in size. In particular embodiments, the size of the nanoparticle has a range from about 300 nm to about 400 nm, including about 300, 310, 320, 330, 340, 350, 360, 370, 380, 390, and 400 nm.

In some embodiments, the presently disclosed particles may comprise other combinations of cationic polymeric blends or block co-polymers. Additional polymers include polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), and polyethylene glycol (PEG). In embodiments, a particle includes blends of other polymer materials to modulate a particle's surface properties. For example, the blend may include non-degradable polymers that are used in the art, such as polystyrene. Thus, in embodiments, a degradable polymer or polymers from above are blended to create a copolymer system. In yet other embodiments, the presently disclosed particle comprises a polymer blend of PBAE, e.g., a mixture of PBAE polymers.

In embodiments, the particles are spherical in shape. In embodiments, the particles have a non-spherical shape. In embodiments, the particles have an ellipsoidal shape with an aspect ratio of the long axis to the short axis between 2 and 10.

In certain embodiments, nanoparticles formed through the presently disclosed procedures that encapsulate active agents, such as a peptide, are themselves encapsulated into a larger nanoparticle, microparticle, or device. In some embodiments, this larger structure is degradable and in other embodiments it is not degradable and instead serves as a reservoir that can be refilled with the nanoparticles. These larger nanoparticles, microparticles, and/or devices can be constructed with any biomaterials and methods that one skilled in the art would be aware. In some embodiments they can be constructed with multi-component degradable cationic polymers as described herein. In other embodiments, they can be constructed with FDA-approved biomaterials, including, but not limited to, poly(lactic-co-glycolic acid) (PLGA). In the case of PLGA and the double emulsion fabrication process as an example, the nanoparticles are part of the aqueous phase in the primary emulsion. In the final PLGA nano- or microparticles, the nanoparticles will remain in the aqueous phase and in the pores/pockets of the PLGA nano- or microparticles. As the microparticles degrade, the nanoparticles will be released, thereby allowing sustained release of the nanoparticles comprising the active agents. In particular embodiments, the nanoparticle or microparticle of the PBAE of formula (I) or formula (II) is encapsulated in a poly(lactic-co-glycolic acid) (PLGA) nanoparticle or microparticle.

As used herein, the term “alkylene” refers to an alkanediyl group having free valencies on adjacent carbon atoms, e.g., —CH(CH₃)CH₂— propylene (systematically called propane-1,2-diyl), including methylene, ethylene, and the like, having between C₁-C₃₀ carbon atoms. Thus, the term “alkylene” by itself or a part of another substituent refers to a straight or branched bivalent aliphatic hydrocarbon group derived from an alkyl group having from 1 to about 30 carbon atoms, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 carbon atoms.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

The term “alkyl” refers to univalent groups derived from alkanes by removal of a hydrogen atom from any carbon atom —CₙH₂ₙ₊₁. The groups derived by removal of a hydrogen atom from a terminal carbon atom of unbranched alkanes form a subclass of normal alkyl (n-alkyl) groups H(CH₂)ₙ. The groups RCH₂, R₂CH (R≠H), and R₃C (R≠H) are primary, secondary and tertiary alkyl groups, respectively.

The term “carbocyclic” refers to cyclic compounds in which all of the ring members are carbon atoms. Representative monocyclic carbocyclic rings include cyclopentyl, cyclohexyl, and cycloheptyl.

The term “heterocyclic” refers to cyclic compounds having as ring members atoms of at least two different elements. Representative heterocyclic compounds include pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like. Non-limiting examples of heterocyclic groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The term “aryl” means, unless otherwise stated, an aromatic hydrocarbon substituent that can be a single ring or multiple rings (such as from 1 to 3 rings), which are fused together or linked covalently. Non-limiting examples of aryl include phenyl, 1-naphthyl, 2-naphthyl, and 4-biphenyl.

### D. Methods for Treating Diarrhea

In some embodiments, the presently disclosed subject matter provides a method for treating a diarrheal disease, the method comprising administering to a subject in need of treatment thereof a therapeutically effective amount of a peptide of sequence SEQ ID NO:1, SEQ ID NO:2, SEQ ID NO:3, OR SEQ ID NO:4 conjugated with a nanoparticle or a ligand as provided hereinabove. In particular embodiments, a peptide of sequence SEQ ID NO:1, SEQ ID NO:2, SEQ ID NO:3, OR SEQ ID NO:4 is conjugated with a branched PBAE of formula (I) or formula (II).

In particular embodiments, the method treats diarrheal diseases in which NHE3 is present, but is inhibited. In other embodiments, the diarrheal disease comprises a diarrheal disease in which NH3 is present, but not inhibited.

Diarrhea can be considered to be acute, persistent, or chronic. Acute diarrhea is short term and can last about one or two days. Persistent diarrhea can last more than two weeks and less than four weeks. Chronic diarrhea generally is defined as having three or more loose stools daily for at least four weeks. Acute diarrhea can be caused by a virus or can arise from ingesting contaminated food or water, e.g., food or water contaminated by bacteria. Persistent diarrhea can result from an ongoing infection lasting between two and four weeks. Chronic diarrhea can be caused by an underlying health condition, such as irritable bowel syndrome. Chronic diarrhea also can be related to an intestinal disease, such as Crohn's disease or celiac disease. Further, some infections, such as a parasitic infection, can cause chronic diarrhea.

The most common causes of acute and persistent diarrhea are infections, traveler's diarrhea, and the side effects of medicines.

Three types of infections that cause diarrhea include viral infections, such as norovirus and rotavirus, including viral gastroenteritis; bacterial infections through contaminated food or water, i.e., foodborne illnesses, including infections caused by common bacteria, such as, Campylobacter, Escherichia coli (E. coli), Salmonella, and Shigella; parasitic infections including Cryptosporidium enteritis, Entamoeba histolytica, and Giardia lamblia.

Traveler's diarrhea is caused by eating food or drinking water contaminated with bacteria, viruses, or parasites and can be a problem for people traveling to foreign countries. Traveler's diarrhea is most often acute. Some parasites, however, cause traveler's diarrhea that lasts longer.

Further, many medicines may cause diarrhea, including antibiotics, anti-hypertensives, antacids containing magnesium, and medicines used to treat cancer.

Some infections, food allergies and intolerances, digestive tract problems, abdominal surgery, and long-term use of medicines can cause chronic diarrhea. For example, some infections from bacteria and parasites that cause diarrhea do not go away quickly without treatment. Also, after an infection, people may have problems digesting carbohydrates, such as lactose or proteins in foods, including cow's milk, milk products, or soy. Problems digesting carbohydrates or proteins can prolong diarrhea.

Allergies to foods such as cow's milk, soy, cereal grains, eggs, and seafood may cause chronic diarrhea. Lactose intolerance is a common condition that may cause diarrhea after eating foods or drinking liquids that contain milk or milk products. Fructose intolerance is a condition that may cause diarrhea after eating foods or drinking liquids that contain fructose, a sugar found in fruits, fruit juices, and honey. Fructose is added to many foods and soft drinks as a sweetener called high-fructose corn syrup. Sugar alcohols such as sorbitol, mannitol, and xylitol may cause diarrhea in some people. Sugar-free candies and gum often include these sugar alcohols.

Digestive tract problems that may cause chronic diarrhea include celiac disease; Crohn's disease; irritable bowel syndrome and other functional gastrointestinal (GI) disorders; small intestinal bacterial overgrowth; ulcerative colitis; and abdominal surgery. Abdominal surgery includes an operation on the appendix, gallbladder, large intestine, liver, pancreas, small intestine, spleen, or stomach.

Long-term use of medicines can cause chronic diarrhea. Some medicines, such as antibiotics, can change the normal gut flora and increase chances of infection with Clostridioides difficile, a bacterium that can cause chronic diarrhea.

The main symptom of diarrhea is passing loose, watery stools three or more times a day. Subjects experiencing diarrhea may also have one or more of the following symptoms: an urgent need to use the bathroom; cramping; loss of control of bowel movements; nausea; and pain in the abdomen. Diarrhea caused by some infections also can give rise to one or more of the following symptoms: bloody stool; fever and chills; light-headedness and dizziness; and vomiting. Diarrhea also can cause dehydration and malabsorption.

Dehydration and malabsorption can be serious complications of diarrhea, especially in infants, toddlers, and young children. Symptoms of dehydration in adults include: thirst; urinating less than usual; feeling tired; dark-colored urine; dry mouth; decreased skin turgor (i.e., when skin is pinched and released, the skin does not flatten back to normal right away); sunken eyes or cheeks; and light-headedness or fainting. Likewise, signs of dehydration in infants, toddlers, and young children include: thirst; urinating less than usual; or no wet diapers for 3 hours or more; a lack of energy; dry mouth; no tears when crying; decreased skin turgor; sunken eyes or cheeks, or a soft spot in the skull.

Symptoms of malabsorption in adults include: bloating; changes in appetite; gas; loose, greasy, foul-smelling bowel movements; and weight loss. Symptoms of malabsorption in infants, toddlers, and young children include: bloating; changes in appetite; gas; loose, greasy, foul-smelling bowel movements; and weight loss or poor weight gain.

Diarrhea can be divided into three categories: watery, fatty (malabsorption), and inflammatory. Watery diarrhea can be further subdivided into osmotic, secretory, and functional types.

Functional watery diarrhea is characterized by chronic or recurrent diarrhea that is not explained by structural or biochemical abnormalities and can include diarrhea resulting from irritable bowel syndrome (IBS), nonceliac gluten sensitivity, and paradoxical diarrhea.

Secretory watery diarrhea can include diarrhea arising from bacterial enterotoxins, e.g., cholera, traveler's diarrhea, bile acid malabsorption, Brainerd diarrhea, Crohn's disease ileitis, an endocrine disorder, medication, alcoholism, microscopic colitis, a neuroendrocrine tumor, vasculitis (e.g., Behçet syndrome) and postsurgical (e.g., gastrectomy, cholecystectomy, vagotomy, or intestinal resection).

Osmotic watery diarrhea can include carbohydrate malabsorption, e.g., lactose, fructose (can also cause a fatty malabsorptive diarrhea); celiac disease (can also cause a fatty malabsorptive diarrhea); medications; and excessive intake of certain foods.

Fatty diarrhea-malabsorptive disorders include amyloidosis, chronic mesenteric ischemia, lymphatic damage (e.g., congestive heart failure, lymphoma), medications (e.g., orlistat, acarbose); noninvasive small bowel parasite (e.g., Giardia); small intestinal bacterial overgrowth, structural (e.g., gastric bypass, short bowel syndrome, and fistulae, tropical sprue; and whipple disease (Tropheryma whippelii infection).

Fatty diarrhea-maldigestive disorders include hepatobiliary disorders, inadequate luminal bile acid; and pancreatic exocrine insufficiency.

Inflammatory diarrhea includes colorectal cancer, inflammatory bowel disease, invasive bacterial infection (e.g., tuberculosis, yersiniosis), invasive parasitic infection, e.g., Entamoeba, ischemic colitis (usually acute, but can be chronic), Pseudomembranous colitis (Clostridioides difficile), radiation enteritis, and ulcerating viral infections (e.g., herpes simplex, cytomegalovirus).

In some embodiments, the diarrhea is associated with cerebrotendinous xanthomatosis (CTx),

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed peptides can be administered prophylactically to prevent or reduce the incidence or recurrence of the disease, disorder, or condition.

In general, the “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; poultry, such as domestic fowls including, but not limited to chickens, turkeys, geese, ducks, quail, guinea fowl, and pigeons; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In particular embodiments, the subject is a human subject. In certain embodiments, the subject is an adult subject. In certain embodiments, the subject is an infant or pediatric subject. In certain embodiments, the subject has an age selected from about 16 years of age or less, about 12 years of age or less, about 8 years of age or less, about 5 years of age or less, and about 2 years of age or less.

In particular embodiments, the subject is an infant. As used herein, the term “infant” can refer to a child from about one month after birth to one year after birth and can include a child up to about two years after birth. As used herein, the term “newborn” or “neonate” refers to an infant in the first 28 days after birth, and can be an infant only a few hours after birth, a few days after birth, or up to a month after birth. The term applies generally to premature, full term (e.g., 38 weeks and beyond), and post mature infants.

In yet more particular embodiments, the human subject is a premature infant. As used herein, the term “premature birth” or “preterm birth” refers to the birth of a baby at fewer than 37 weeks gestational age and can include babies born at 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, 24 weeks, 23 weeks, 22 weeks, 21 weeks, and 20 weeks gestational age.

In some embodiments, administering a therapeutically effective amount of the peptide treats one or more symptoms of the diarrheal disease. In certain embodiments, administering a therapeutically effective amount of the peptide shortens a duration of the diarrheal disease, lessens a volume of diarrhea, and combinations thereof.

In certain embodiments, administering a therapeutically effective amount of the peptide increases intestinal Na⁺ absorption, rehydrates the subject, and combinations thereof.

In certain embodiments, administering a therapeutically effective amount of the peptide stimulates sodium-hydrogen exchanger 3 (NHE3) activity, prevents or diminishes CAMP inhibition of NHE3 activity, reverses reduced NHE3 activity caused by elevated CAMP, cGMP, and Ca²⁺, and inflammatory causes of diarrhea, and combinations thereof. In certain embodiments, administering a therapeutically effective amount of the peptide prevents changes in net water secretion caused by two major bacterial enterotoxins responsible for cholera and Traveler's diarrhea.

In certain embodiments, administering a therapeutically effective amount of the peptide stimulates sodium-hydrogen exchanger 3 (NHE3) activity, prevents or diminishes CAMP inhibition of NHE3 activity, reverses reduced NHE3 activity caused by elevated CAMP, cGMP, and Ca²⁺, and inflammatory causes of diarrhea, and combinations thereof, in a small intestinal loop of the subject. In particular embodiments, the small intestinal loop of the subject includes a jejunum and/or an ileum of the subject.

In particular embodiments, the peptide is delivered to a gastrointestinal tract (GI) tract of the subject. In more particular embodiments, the peptide is deposited subapically in the GI tract.

The term “combination” is used in its broadest sense and means that a subject is administered at least two agents, more particularly a peptide described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compositions described herein can be administered alone or in combination with adjuvants that enhance stability of the compositions, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a peptide described herein and at least one additional therapeutic agent can be varied so long as the beneficial effects of the combination of these agents are achieved. Accordingly, the phrase “in combination with” refers to the administration of a peptide described herein and at least one additional therapeutic agent either simultaneously, sequentially, or a combination thereof. Therefore, a subject administered a combination of a peptide described herein and at least one additional therapeutic agent can receive a peptide and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the peptide described herein and at least one additional therapeutic agent are administered simultaneously, they can be administered to the subject as separate pharmaceutical compositions, each comprising either a peptide or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a peptide described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

A Novel Peptide Prevents Enterotoxin- and Inflammation-Induced Intestinal Fluid Secretion by Stimulating NHE3 Activity

### 1.1 Overview

In this Example, a peptide (N3SP) was synthesized to mimic the part of the NHE3 C-terminus that forms a multi-protein complex that inhibits NHE3 activity. The effect of N3SP on NHE3 activity was evaluated in NHE3 transfected fibroblasts null for other plasma membrane NHEs, a human colon cancer cell line that models intestinal absorptive enterocytes (Caco-2/BBe), human enteroids, and mouse intestine in vitro and in vivo. N3SP was delivered into cells via a hydrophobic fluorescent maleimide or by nanoparticles.

The presently disclosed results demonstrate that N3SP uptake stimulated NHE3 activity at nM concentrations under basal conditions and partially reversed the reduced NHE3 activity caused by elevated cAMP, cGMP, and Ca²⁺ in cell lines and in in vitro mouse intestine. N3SP also stimulated intestinal fluid absorption in the mouse small intestine in vivo and prevented cholera toxin-, E. coli heat stable enterotoxin-, and CD3 inflammation-induced fluid secretion in a live mouse intestinal loop model. These findings suggest pharmacologic stimulation of NHE3 activity as an efficacious approach for the treatment of moderate/severe diarrheal diseases.

### 1.2 Background

The epithelial BB Na⁺/H⁺ antiporter (NHE3) accounts for a major fraction of intestinal and renal Na⁺ absorption by taking part in a process called neutral NaCl absorption. Zachos et al., 2016; Rao, 2019; Zachos et al., 2005. In this process, NHE3 is linked to BB Cl⁻/HCO₃⁻ exchangers of the SLC26A gene family, thought to be primarily SLC26A3 (Walker et al., 2008; Xia et al., 2014; Musch et al., 2009; Haggie et al., 2018).

NHE3 is highly regulated as part of digestion, initially being inhibited, which contributes to the spreading of digestive enzymes over the absorptive/digestive surface, and then stimulated later in digestion, which helps avoid post-prandial dehydration (Zachos et al., 2016; Zachos et al., 2005; Kapus et al., 1994; Levine et al., 1993; Camilleri et al., 2017; Barrett, 2020). In almost all diarrheal diseases, NHE3 activity is inhibited, mimicking the early stages of digestion, although with a prolonged time course (Camilleri et al., 2017; Barrett, 2020).

The cyclical inhibition/stimulation of NHE3 occurs via two large signaling complexes that form on its intracellular regulatory domain (approximately aa 455-832) (Kapus et al., 1994; Levine et al., 1993; Donowitz and Li, 2007; Donowitz et al., 2009; Sarker et al., 2008; Akhter et al., 2002; Hendus-Altenburger et al., 2014). One complex is involved in acute stimulation and the other complex is involved primarily in acute inhibition.

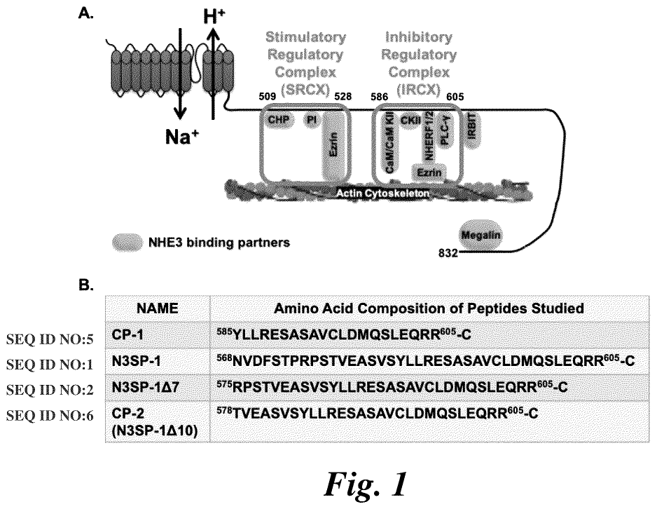

Previous studies conducted by us and others, have defined a region of the NHE3 C-terminus (aa 586-605) that interacts primarily with proteins that inhibit NHE3 activity; although some NHE3 stimulatory proteins bind in this area, as well (Levine et al., 1993; Sarker et al., 2008; Akhter et al., 2002; Hendus-Altenburger et al., 2014; Zizak et al., 2012). Significance of this site has been confirmed by mutational studies, and interactions validated by co-precipitation experiments, and in vitro binding studies with NHE3 C-terminus truncated to aa 605, but not aa 585. Proteins that interact with NHE3 at this site include CaMKIIδ, CK2, NHERFs 1-4, PLCγ, and CaM (FIG. 1A), while IRBIT binds to NHE3 between aa 585-689.

Without wishing to be bound to any one particular theory, it was thought that a NHE3 mimetic peptide based on this largely inhibitory region might be a putative competitor of protein-protein interactions of the inhibitory regulatory complex, which would result in pharmacologic stimulation of NHE3 activity. An ideal mimetic peptide should: (i) be cell permeant, (ii) work at low concentrations, (iii) increase basal NHE3 activity with or without inhibiting cAMP, cGMP, calcium- and inflammation-mediated inhibition of NHE3 activity, and (iv) alter Na⁺/H⁺ activity in a specific manner. If successful, this mimetic could be a candidate drug for treating diarrheal diseases.

### 1.3 Methods and Materials

### 1.3.1 NHE3 Inhibitory Domain (FIG. 1A) Mimicking Peptides

Synthesized by Peptide2.0 (Chantilly, VA) and purity assessed to be greater than 98% by HPLC. Peptides include (see FIG. 1B): (a) control peptide-1 (CP-1); (b) NHE3 stimulatory peptide-1 (N3SP-1); (c) N3SP-1Δ7; (d) CP-2 (N3SP-1A10). A C-terminal cysteine was added to the peptides for conjugation to BODIPY 577/618. For nanoparticle uptake studies, an N-terminal biotin was added to CP-2 and N3SP-1Δ7.

### 1.3.2 Reagents/Materials

BODIPY 577/618 maleimide, nigericin, 2′,7′-Bis(carboxyethyl)-5-6-carboxyfluorescein-AM (BCECF), 1,4 (and 5)-benzenedicarboxylic acid, 2-[10-(dimethylamino)-4-fluoro-3-oxo-3H-benzo[c]xanthen-7-yl](carboxy SNARF-4F), Alexa-Fluor-conjugated streptavidin, Hoescht 33342, and Alexa-Fluor-conjugated mouse monoclonal and rabbit polyclonal secondary antibodies were from Life Technologies. IRDye monoclonal and polyclonal secondary antibodies were from Rockland, Inc. Monoclonal anti-hemagglutinin (HA) affinity matrix was from Roche Diagnostics (Cat #: 1181501600). Rabbit polyclonal human NHE3 antibody was from Novus, Inc (Cat #: NBP1-82574). Hamster anti-murine CD3 mAb (2C11) from Pharmingen (San Diego, California) was a gift from Dr. Jerrold Turner (Brigham and Women's Hospital).

1.3.3 Nanoparticle materials

The following monomers were purchased from Sigma-Aldrich (St. Louis, MO): bisphenol A glycerolate diacrylate (B7) (CAS 4687-94-9), trimethyl propane triacrylate (B8), (CAS 15625-89-5), and 1,3-diaminopropane (E1) (CAS 109-76-2). 4-amino-1-butanol (S4) (CAS 13325 October 5) was purchased from Alfa Aesar (Ward Hill, MA). Bioreducible base monomer 2,20-disulfanediylbis(ethane-2,1-diyl)diacrylate (BR6) was synthesized as described by Kozielski et al., 2013. Carboxylate ligand C5 was synthesized as described by Rui et al., 2019.

### 1.3.4 Cells

All immortalized cell lines and heterologous expression of NHE3 have been described previously (Zacho et al., 2005; Levine et al., 1993). Briefly, PS120 cells (a fibroblast cell line lacking all endogenous plasma membrane NHEs) were stably transfected with triple HA-N-terminally tagged rabbit NHE3 (HA3-NHE3) constructs. Polarized Caco-2/BBe cells (a human colon cancer cell line with small intestinal properties), which express NHERFs 1-4, but undetectable NHE3 activity, were transduced on day 12 post-confluency with an adenovirus-triple HA-tagged NHE3 construct and studied 48 h post infection. Human jejunal enteroid cultures were established from deidentified normal, healthy adult subjects, as previously described (Zachos et al., 2016; Sunuwar et al., 2020; Foulke-Abel et al., 2020; Yin et al., 2021), with JHU IRB approval (NA_0038329). Human enteroids, which express BB NHE3, were cultured in Matrigel (Corning Cat #: 356231) and studied as confluent monolayers on collagen IV-coated Transwell inserts (Corning Cat #: 3470), as described (Noel et al., 2017; Staab et al., 2020). 1.3.5 Mice 8-12 week old male and female C57B1/6N mice were starved overnight and provided water ad libitum prior to experiments that consisted of: (a) sacrifice and acute removal of jejunum for studies by multi-photon measurement of basal NHE3 activity, as previously reported (Murtazina et al., 2011); (b) cannulation for measurement of basal in vivo net water transport by perfusion using the non-absorbable marker, Fe (CN) 6 (Clayburgh et al., 2006); or (c) formation of upper small intestinal 3-4 cm loops for study of baseline fluid absorption or cholera toxinor E. coli heat stable enterotoxin-induced fluid secretion and intestinal fluid secretion induced by IP anti-CD3 monoclonal antibody injection (Clayburgh et al., 2006). All experimental protocols performed in mice were approved by the Institutional Animal Care and Use Committee of the Johns Hopkins University (MO19M118).

### 1.3.6 In Vitro Transport Assays Cells/Enteroids/Mouse Intestine

NHE3 activity in PS120, Caco-2/BBe cells and human jejunal enteroids was determined fluorometrically (Photon Technologies Incorporated) using the intracellular pH sensitive dye BCECF, as reported (Levine et al., 1993; Sarker et al., 2008; Foulke-Abel et al., 2020). PS120/HA3-NHE3 cells were seeded on glass cover slips and studied at 70-80% confluency. Caco-2/BBe/HA3-NHE3 cells were grown to confluency on Transwell inserts (polyester, 0.4-μm pore size) and studied approximately 14-16 days pod. Human duodenal enteroid monolayers on Transwell inserts were differentiated for 5 days in the absence of Wnt3a, R-spondin, and noggin as described (Noel et al., 2017; Staab et al., 2020).

Rates of Na⁺/H⁺ exchange were determined as Na⁺-dependent alkalinization in the presence of 50-μM HOE-694 (to inhibit all other plasma membrane NHE isoforms) in HCO₃-free solutions, with internal calibration using the nigericin/K+ method, as previously described (Sarker et al., 2008; Foulke-Abel et al., 2020). Kinetic analysis was used for PS120 cell studies (expressed as Vₘₐₓ) and initial rates (ΔpH/min) for Caco-2/BBe cells, mouse intestine in vitro, and human enteroids. For in vitro mouse jejunal transport studies, NHE3 activity (in the presence of 50-μM HOE-694) was measured using SNARF-4F at 37° C. in a perfusion chamber using a two-photon microscope (Olympus FV1000) and 25× objective, as described (Murtazina et al., 2011).

In vitro determination of electrogenic active ion transport in mouse jejunum was performed by Ussing Chamber/voltage clamp technique, as described (Tse et al., 2018). For the measurement of active anion secretion using the Ussing chamber/voltage clamp technique, mice were sacrificed and intact jejunum was mounted between two halves of an Ussing chamber for measurement of short-circuit current (Isc), potential difference (PD), and transepithelial electrical resistance (TER). These measurements were performed at 37° C. with tissue exposed to a solution (gassed with 5% CO₂/95% O₂) containing 140-mM NaCl, 5-mM KCl, 1-mM MgCl₂, 2-mM CaCl₂), 10 mM HEPES with 10-mM D-glucose on the mucosal surface and 10-mM mannitol on the serosal surface. Tissue was continually voltage clamped to zero PD except for short periods to determine the PD. Tissues were allowed to reach steady state values followed by sequential addition to the serosal surface of 10-μM forskolin (FSK) and 10-μM carbachol (CCH) with determination of peak increase in Isc caused by each.

### 1.3.7 In Vivo Transport Assays

All in vivo studies were performed on 8-12 week old C57B1/6N mice anesthetized with isofluorane and kept at 37° C. via heating lamps and rectal thermal probes.

### 1.3.8 BODIPY-Conjugated Peptide Studies

To measure peptide effects on basal net water transport, 3-4 cm loops were prepared with: (a) in-flow and outflow catheters. Loops were perfused (1 mL/min with peristaltic pump) with 90-mM NaCl, 25-mM NaHCO₃, 30-mM HEPES, 5-mM KCl, 1.2-mM CaCl₂), 20-mM glucose, pH 7.4. containing 2-mM Na ferrocyanide as a non-absorbable marker with CP or N₃. 30 min stabilization period was followed by three 20 min perfusion periods. Ferrocyanide concentration was determined by colorimetric assay (Clayburgh et al., 2006) or; (b) 150 1 PBS (pH 7.4, 100-mM NaCl, 25-mM NaHCO₃, 5-mM KCL, 1.2-mM CaCl₂, 30 mM HEPES) with control or test peptide (400 nM) was incubated for 4 h, the loops drained and 150 μL PBS added; after 30 min the animals were sacrificed, the loops removed, weighed and wet weight/length determined. Cholera toxin (CTx)-induced fluid secretion was determined on 3-4 cm small intestinal ligated loops (starting approximately 2-cm distal to the Ligament of Treitz) with instillation of 100 μL of PBS with or without 0.1-μg purified CTx (Sigma) with 400-nM of CP-1, N3SP-1, or PBS alone. The peptides were conjugated to BODIPY. After loop preparation, abdomens were closed, mice removed from anesthesia, and recovered in separate cages for the next 6 hours. After this time, animals were sacrificed by cervical dislocation, intestinal segments removed, weighed and length determined. Data are presented as loop weight/cm. Similar studies were performed with instillation of E. coli heat stable enterotoxin (0.05 μg) studied for 4 hours. In the anti-CD3 antibody experiments, jejunal loops were injected with 100 μL of PBS, CP-1 (400 nM/100 μL) or N3SP-1 (400 nM/100 μL); immediately after the abdomens were closed surgically, 200 μg of anti-CD3 monoclonal antibody in 200-μL PBS vs 200-μL PBS alone were injected IP. The animals were sacrificed 2.5 hours after the IP injections and loop weight/cm length determined (Clayburgh et al., 2006).

### 1.3.9 Nanoparticle-Conjugated Peptide Studies

Effect on basal fluid transport and effect of Tenapanor. Mice operated on as above had 3-4 cm closed ileal loops inoculated with 150-μL PBS containing 400-nM nanoparticle-CP-2 or -N3SP-1Δ7. After 4 h, the loops were drained and inoculated with 150-μL PBS. 30 min later, the animals were sacrificed and loop weight/length determined. In parallel studies, Tenapanor (10 μM) was present in both incubation periods. Similar to the BODIPY-conjugated peptide studies, the effects of nanoparticle-CP-2 or N3SP-1Δ7 (4 μM) compared to PBS alone were determined on net fluid secretion produced by inoculation of 0.1-μg CTx. The nanoparticle-peptides plus 0.1-μg CTx in 150-μL PBS were inoculated in 3-4 cm long closed ileal loops. Additional controls included loops exposed only to 150-μL PBS and those only exposed to the CTx/PBS. 4 h later, the animals were sacrificed and loop weight/cm length was determined.

### 1.3.10 Immunofluorescence/Confocal Microscopy

Mouse jejunum was fixed in 10% neutral buffered formalin, processed, and paraffin-embedded. 5-μm sections were washed in xylene and rehydrated in gradient ethanols. Sections were exposed to antigen retrieval in 10-mM sodium citrate (pH 6.0) for 10 min, washed in ddH₂0, and then blocked in buffer containing 2% bovine serum albumin, 15% fetal bovine serum, and 0.1% saponin for 30 min. Anti-NHE3 antibody (1:100) was incubated in blocking buffer overnight and then washed three times in PBS. Sections were then exposed to AlexaFluor-conjugated secondary antibody (1:100) and Hoescht 33342 for 1 hour, washed in PBS, mounted and coverslipped. Confocal images were obtained on a Zeiss 510 META confocal microscope or Olympus Flow View3000RS using 63× water or 40× oil immersion objectives (UPLSAPO 100xs 1.35 NA silicone objective), respectively.

1.3.11 cAMP Assay

Human jejunal enteroid monolayers on Transwell inserts (Corning Cat #: 3470) were differentiated for 5 days and then in serum-free media were exposed apically to nanoparticles containing either 250-nM CP-2 or N3SP-1Δ7. The peptide and polymer in PBS were prepared just before use, allowed to combine for 10 min before being added to the apical surface of enteroid monolayers at room temperature. Monolayers were incubated for 4 hours at 37° C., 5% CO₂ incubator before the nanoparticles were removed, then monolayers were washed 3 times with cold PBS and changed to differentiation media and moved to a 37° C. incubator. Differentiation media was removed 24 hours later and washed twice with CMGF-media (Advanced DMEM/F12 with 10-mM Hepes, 10-mM GlutaMax, and 1000 Units/mL Penn/Strep). Next, 100 μL of CMGF-media containing 10-mM Theophylline, 50-μM Cilostazol and 50-μM Rolipram was added to the apical and basolateral surfaces followed by exposure to FSK (10 μM) at 37° C., 5% CO₂, for 45 min before apical and basolateral media were collected and cells were lysed in 200-μL 0.1 M HCl for cAMP analysis. CAMP in apical, basolateral, and cells was determined using nonradioactive, CAMP-ELISA kits from ENZO Life Sciences (Loerrach, German), using the acetylation protocol in which the yellow-colored product is inversely proportional to the amount of CAMP present in the sample.

### 1.3.12 BODIPY-Peptide Conjugation

BODIPY maleimides are hydrophobic and demonstrated by us and others to facilitate entry of small peptides into cells (Zachos et al., 2009; Boehning et al., 2005). Briefly, peptides were reconstituted in PBS and exposed to TCEP (1 mM) and BODIPY under inert conditions for 2 hours at room temperature with constant end-over-end rotation. Unconjugated BODIPY was removed from the solution using sulpho-agarose beads for 2 hours at room temperature with constant rotation. After pelleting beads by gravity, supernatant containing BODIPY-conjugated peptides was stored at 4° C. and used within 1 week of preparation.

### 1.3.13 Nanoparticle Preparation

### 1.3.13.1 Polymer Synthesis

Hyperbranched poly(beta amino-ester) (PBAE) was synthesized as previously described by Rui et al., 2019. Briefly, monomers BR6, B7 and B8 were combined at a 0.16:0.64:0.2 molar ratio (BR6: B7: B8) in anhydrous DMF. Monomer S4 was added at a 2.2:1 vinyl: amine ratio, with a final monomer concentration of 150 mg/mL. Polymerization proceeded overnight at 90° C. with stirring. The resulting acrylate-terminated polymers were then endcapped with monomer E1, purified by two diethyl ether washes, followed by a second round of endcapping with monomer C5. Both reactions were performed at a final endcap concentration of 0.2 M for 2 hours at room temperature. The resulting carboxylated polymers were precipitated by diethyl ether and remaining solvent was removed in a desiccation chamber under vacuum. The polymer was dissolved in anhydrous DMSO to a final concentration of 100 mg/mL and stored with desiccant at −20° C.

### 1.3.13.2 Polymer Characterization

Polymer structure was characterized by nuclear magnetic resonance spectroscopy (NMR) via 500 MHz 1H NMR in CDCl₃ (Bruker, Billerica, MA) and analyzed using the TopSpin 3.5 software. The presence of acrylate groups in the acrylate-terminated base polymer was confirmed by peaks in the 6-6.5 ppm range, which disappeared upon polymer endcapping. Gel Permeation Chromatography (GPC) (Waters, Milford, MA) was used to characterize polymer molecular weight relative to polystyrene standards.

### 1.3.13.3 Nanoparticle Synthesis and Characterization

PBAE polymer and peptide were separately dissolved in pH 7.4 150-mM PBS and combined at a 1:1 volume ratio. Final concentrations of the components were 6-ng/μL peptide and 180-ng/μL polymer (30:1 polymer: peptide weight ratio). Polymer and peptide nanoparticles were allowed to self-assemble at room temperature for 10 minutes. For characterization, nanoparticles were diluted 1:5 in PBS. Hydrodynamic diameter and zeta potential were measured using a Malvern Zetasizer Pro (Malvern Panalytical, Malvern, UK). Stability was assessed by incubating nanoparticles at RT up to 45 minutes then measuring hydrodynamic diameter over 45 minutes. Nanoparticles were made immediately prior to each experiment.

### 1.3.13.3.1 TEM Morphology of Nanoparticle-Peptides by Transmission Electron Microscopy

2-μM PBAE nanoparticle-peptide in PBS were applied to a square copper 400-mesh grid (Electron Microscopy Sciences, Hatfield, PA). Samples were air-dried overnight at room temperature. Samples were then dipped in a 0.5-wt % uranyl acetate solution and airdried for 1 hour at room temperature. Once samples were fully dried, the mesh grid was imaged on a Hitachi 7600 transmission electron microscope (Hitachi High-Technologies, Tokyo, Japan).

### 1.3.13.3.2 Effects of pH on Nanoparticle Viability and Uptake

B16F10 cells were plated at 15,000 cells/well of a 96-well plate in RPMI 1640 medium with 5% fetal bovine serum and 1% penicillin/streptomycin (Thermo Fisher Scientific, Waltham, MA). 24 hours after plating, media were changed and replaced with serum-free RPMI 1640. PBAE NPs encapsulating fluorescent peptide or fluorescent peptide alone at 2-μM concentration in PBS were exposed to varying pH conditions (pH 1.2, pH 4, and pH7) for 15 minutes. After 15 minutes, samples were neutralized to pH 7 and added to cells. Samples were incubated with cells at 37° C. for 2 hours. After 2 hours, media was replaced. At 24 hours, cell viability was measured via CellTiter96 MTS assay (Promega, Madison WI). Samples were normalized to a cell only control. At 48 hours, cells were trypsinized, washed, and resuspended in FACS buffer (PBS with 1% FBS). Cells were then measured for uptake via flow cytometry using an Attune NxT cytometer (Thermo Fisher Scientific, Waltham, MA). Uptake was measured as the percentage of cells positive for fluorescent peptide as well as the geometric mean fluorescence of cells. Gating was determined using a cell only negative control.

### 1.3.13.3.3 Effects of pH on Nanoparticle-Peptide Sizing by Dynamic Light Scattering (DLS)

Nanoparticle-peptide (2 μM) in PBS was exposed to varying pH conditions (pH 1.2, pH 4, and pH7) for 15 minutes. After 15 minutes, samples were neutralized to pH 7. Particle size and distribution were then immediately measured via dynamic light scattering (DLS) using a Zetasizer Pro (Malvern Panalytical, Malvern, UK).

### 1.3.13.3.4 Nanoparticle-Peptide Uptake in B16F10 Cells

B16F10 cells (Kozielski et al., 2013) were seeded at 10,000 cells/well in a 96-well plate. Biotinylated N3SP-1Δ7 was conjugated to streptavidin-AlexaFluor 488 and used to synthesize nanoparticles as described. B16-F10 cells were treated with nanoparticles or free peptide at a concentration of 0.25-2 μM peptide dose for multiple times up to 4 h, then washed three times with PBS and replenished with fresh media. The following day, peptide uptake was quantified by measuring AlexaFluor 488 fluorescence on a BD Accuri™ C6 Flow Cytometer (BD Biosciences, San Jose, CA) connected to a HyperCyt™ autosampler (IntelliCyt Corporation, Albuquerque, NM). All flow cytometry data were analyzed using FlowJo software (BD Biosciences, San Jose, CA).

### 1.3.13.3.5 Nanoparticle-Peptide Uptake in Enteroids

BODIPY and nanoparticle conjugated peptides were loaded into cells/enteroids/mouse intestine by conjugation with maleimide or with nanoparticles. BODIPY-conjugated peptides were exposed to the cells and apical surface of polarized cells and lumen of intestinal loops.

50 μL of nanoparticles were added to the apical side of enteroids in fresh serum-free media for a final concentration of 250-nM peptide. After varying times from 15 min to 4 hours, enteroids were washed three times with PBS to remove extracellular nanoparticles and wells were replenished with fresh serum-containing media.

At varying times after nanoparticle addition up to overnight, enteroids were serum-starved for 2 hours then fixed in 4% paraformaldehyde for 30 minutes. After washing with PBS, formaldehyde was neutralized with 20-mM glycine. Enteroids were then permeabilized in 0.1% saponin, 1% BSA for 30 minutes. Fixed and permeabilized enteroids were stained for biotinylated peptide using streptavidin conjugated AlexaFluor 488 (1:100) and Hoechst stain (1:200). Enteroids were thoroughly washed with PBS and mounted onto microscope slides. Uptake was visualized using an Olympus FV3000RS confocal microscope and 40× objective (Olympus, Tokyo, Japan).

### 1.3.13.4 Structural Modeling of Human NHE3 C-Terminus and NHE3 Peptides

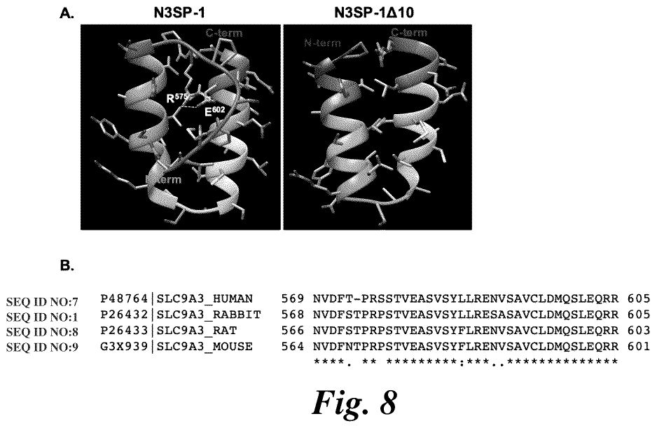

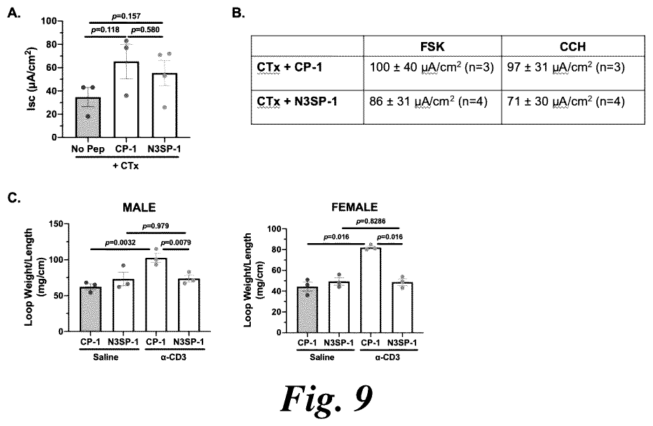

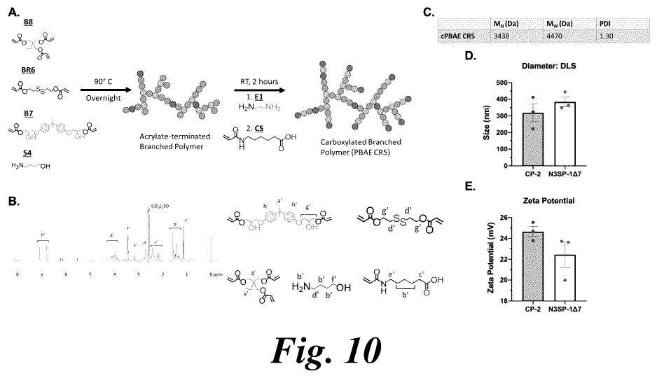

Rosetta (Bender et al., 2016; Rohl et al., 2004; Bonneau et al., 2002) was used to predict the structure of the human NHE3 C-terminus and NHE Peptides. Structural fragment libraries extracted from the Protein Data Bank for NHE3 sequence regions 585-605 (CP-1) and 568-605 (N3SP-1) and several N-terminal truncations including 578-605 (N3SP-1A10) (FIG. 8A) were generated and used to perform a low-resolution conformational search followed by a full-atom model. Ten thousand models were generated for each peptide and the top 10% of lowest energy models were clustered as previously described (Bender et al., 2016). A similar approach was used to model single amino acid deletions from the N3SP-1 N-terminus. Models representing the most frequently sampled conformations for each peptide were selected for presentation.

### 1.3.13.5 Statistical Analysis

Statistical significances were calculated by paired or unpaired t-tests (where indicated) or multiple comparisons ANOVA and were assessed to compare groups including a minimum of n=3 replicates. p values are indicated in figures and figure legends.

### 1.4 Results

### 1.4.1 Computational Modeling and Engineering of NHE3 Mimetic Peptides

We previously defined that aa 586-605 (rabbit NHE3) of the intracellular C-terminal domain is the region that directly binds proteins that inhibit NHE3 activity. We termed this the NHE3 Inhibitory Regulatory Complex (IRCX) (FIG. 1A) (Donowitz and Li, 2007; Donowiwtz et al., 2009; Akhter et al., 2002).

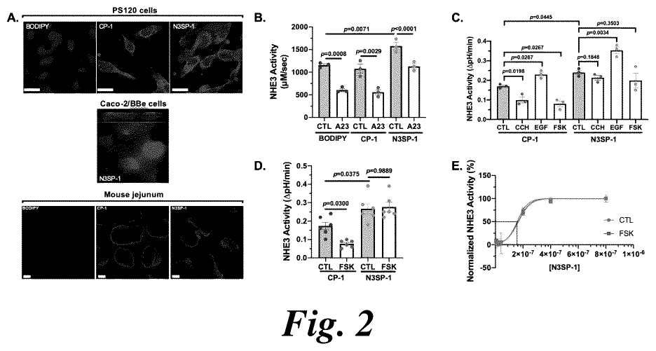

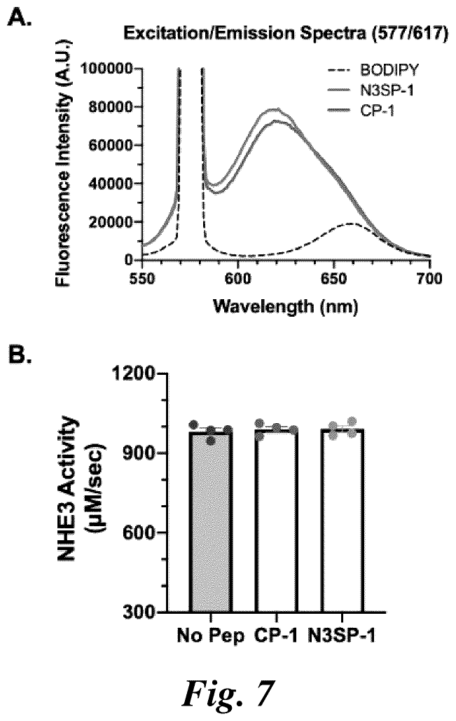

Without wishing to be bound to any one particular theory, it was thought that a peptide mimic of this region would competitively bind proteins of the NHE3 IRCX, reduce their association with NHE3, and prevent NHE3 inhibition. To test this hypothesis, we synthesized a 21 aa peptide representing aa 585-605 of rabbit NHE3 (CP-1; FIG. 1B) with an additional C-terminal Cys to conjugate BODIPY 577/618, a fluorescent maleimide that when conjugated to a peptide/protein renders the peptide cell-permeable and fluorescent at the wavelengths studied (FIG. 7A) (Zachos et al., 2009; Boehning et al., 2005). CP-1 was exposed to PS120/HA3-NHE3/NHERF2 cells and peptide entry confirmed by confocal microscopy (FIG. 2A). There was no significant effect, however, of CP-1 (400 nM) compared to BODIPY alone on basal NHE3 activity (FIG. 8B).

A bioinformatics approach was used to provide insight into aa immediately upstream or downstream from the CP-1 boundaries, considering that adjacent aa might allow increased competition with the IRCX for binding regulatory proteins. Models of the three-dimensional structure of the sequence of the NHE3 C-terminus around the domain involved in binding members of the NHE3 IRCX were created using the Rosetta structural modeling software (see Methods (Bender et al., 2016; Rohl et al., 2004; Bonneau et al., 2002).

Extending the N-terminal sequence of CP-1 to aa 568, while keeping the C-terminal aa 605, predicted a stable structure composed of two alpha helixes with H⁺ bonding between R575 and D602 that stabilized the helices (FIG. 8A). This peptide was named N3SP-1. Removing up to 7 amino acids from the N-terminus of this peptide preserved the ability to stimulate NHE3 activity (Table 2). Removal of 8 or more aa was predicted to lose the hydrogen bonding and result in peptide instability (shown for N3SP-1Δ10 in FIG. 8A) and loss of NHE3 stimulatory activity (Table 2). Multiple sequence alignments of NHE3 from multiple species was generated demonstrating preservation of aa in this area of NHE3 (FIG. 8B).

TABLE 2
Effect of N-terminal Truncations of N3SP-1
on NHE3 Activity in Human Duodenal Enteroids

| | Stimulation of | |
|---|---|---|
| N3SP-1 Variant | NHE3 Activity | EC₅₀ |
| N3SP-1 | YES (38%) | 152 nM |
| N3SP-1Δ18 | NO | |
| CP-2(N3SP-1Δ10) | NO | |
| N3SP-1Δ9 | NO | |
| N3SP-1Δ8 | YES (9%) | 356 μM |
| N3SP-1Δ7 | YES (39%) | 156 nM |
| N3SP-1Δ6 | YES (40%) | 151 nM |
| N3SP-1Δ5 | YES (39%) | 154 nM |

Concentration dependent effects on NHE3 basal activity was determined using BCECF-fluorometry. Kinetic analysis was determined of extent of stimulation of basal NHE3 activity; analysis was by use of the Hill equation. Truncation of 5,6,7 N terminal aa did not significantly alter the magnitude of the stimulation of NHE3 or alter the EC₅₀. Truncation of 8 aa, however, reduced the stimulatory effect and significantly increased the EC₅₀, while further truncations of 9, 10 and 18 aa produced peptides that were without effect on basal NHE3 activity.

### 1.4.2 Functional Characterization of NHE3 Mimetic N3SP-1 Compared to CP-1

### 1.4.2.1 In Vitro Studies-BODIPY Conjugates (FIG. 2A-E)

BODIPY-conjugated N3SP-1 was assayed for effects on NHE3 activity when loaded into PS120/HA3-NHE3/NHERF2 cells (FIG. 2B), Caco-2/BBe/HA3-NHE3 cells (FIG. 2C), and in vitro mouse jejunum (FIG. 2D). Intracellular loading was documented by confocal microscopy (FIG. 2A). CP-1 was used as the negative control in these studies. In all models, N3SP-1 (400 nM) stimulated basal NHE3 activity compared to CP-1 (FIG. 2B-FIG. 2D). No stimulatory effect was detected with exposure to unconjugated N3SP-1 (FIG. 7B). The range of stimulation was 41-61% (PS120=61%, FIG. 2B; Caco-2/BBe=41%, FIG. 2C; and mouse jejunum=57%, FIG. 2D). Increased basal NHE3 activity in Caco-2/BBe/HA3-NHE3 cells was concentration dependent with maximal effect at 400 nM and an EC₅₀ of 152 nM (FIG. 2E).

Further studies examined the effect of N3SP-1 on acute NHE3 inhibition by Ca²⁺ ionophore 4-Br-A23187, FSK, and carbachol, as well as acute stimulation by epidermal growth factor (EGF). N3SP-1 pretreatment prevented inhibition of NHE3 by 0.5-μM A23187 in PS120 fibroblasts (FIG. 2B); FSK (10 μM) in Caco-2/BBe cells and mouse jejunum in vitro (FIG. 2C, FIG. 2D); and carbachol (10 μM) in Caco-2/BBe cells (FIG. 2C); but did not alter EGF (200 ng/mL) stimulation in Caco-2/BBe cells (38% vs 35% stimulation in presence of CP-1 vs N3SP-1, respectively; FIG. 2C). The loss of FSK inhibition on NHE3 activity in Caco-2/BBe cells had a similar concentration dependent response to that on basal NHE3 activity, with EC₅₀ of 157 nM after addition apically to Caco-2/BBe cells (FIG. 2E). These results demonstrate that N3SP-1, but not CP-1, prevents CAMP- and Ca²⁺-mediated inhibition of NHE3 activity in vitro. We next determined whether N3SP-1 exerts similar effects in vivo.

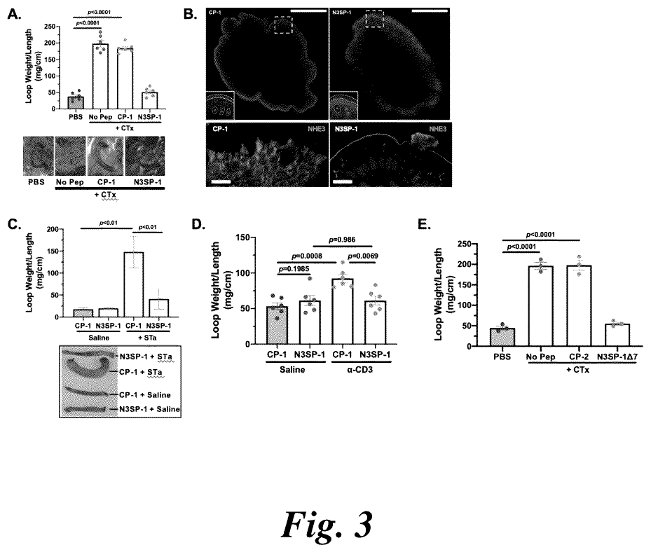

### 1.4.2.2 In Vivo Studies-BODIPY Conjugates (FIG. 3A-E)

The effect of the N3SP-1 was determined in live mouse models, measuring net water transport under basal conditions and in two bacterial enterotoxin-induced (i.e., Cholera toxin (CTx) and E. coli heat stable (Sta) enterotoxin) diarrheal disease closed loop models. Acute exposure of mouse jejunum to N3SP-1 (400 nM) via intraluminal perfusion (Clayburgh ct al., 2006) acutely stimulated basal water absorption compared to CP-1. In a typical experiment, jejunal net water transport was 4.2 μL/min/cm with CP-1 and 50.3 L/min/cm with N3SP-1; the difference in net water transport between N3SP-1 and CP-1 perfused jejunal loops was 56.6±15.1 μL/min/cm (n=5; p=0.0004).

In the closed loop model, purified CTx (1 μg/3-4 cm loop) was instilled in the presence of 400-nM N3SP-1 or CP-1 for 6 hours and then net fluid secretion (loop weight/cm) was determined (FIG. 3A). N3SP-1 prevented CTx-stimulated fluid secretion, while CP-1 did not alter fluid accumulation compared to CTx alone (FIG. 3A). This effect of N3SP-1 on CTx-induced secretion was associated with visible intracellular accumulation of the peptide after 6 hours of exposure and contrasted to the localization of CP-1, which remained at the BB (FIG. 3B). This difference in peptide localization was associated with differential localization of NHE3. By confocal microscopy (FIG. 3B), NHE3 was internalized in the CTx-treated mouse jejunum when exposed to CP-1. In contrast, NHE3 was detected on the CTx-treated jejunal BB after exposure to N3SP-1. These results suggest that reduced luminal fluid accumulation in CTx-treated mouse intestinal loops by N3SP-1 is associated with increased BB expression of NHE3 compared to CP-1.

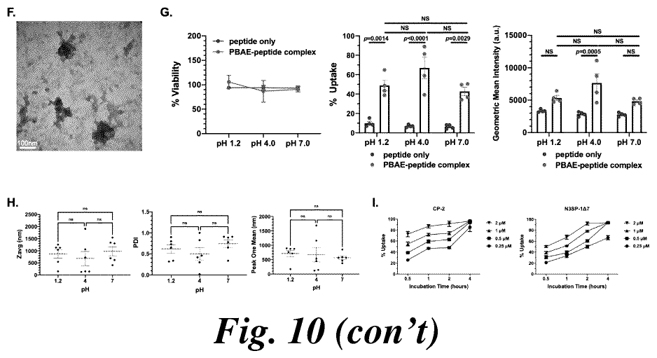

The N3SP-1 effect so dramatically reduced the CTx-induced fluid secretion that specificity of the effect was determined, in spite of the fact that N3SP-1 is part of the NHE3 C-terminus and is not matched by any other protein sequence, as assessed by BLAST. This determination was done by measuring the short-circuit current (Isc) in loops exposed to CTx for 6 hours in the presence of PBS, CP-1, or N3SP-1 added at the same time as CTx (FIG. 9A). Changes in Isc were then determined in treated loops after sequential exposure to basolateral FSK followed by carbachol with peak Isc determined following exposure of each agonist. The assumption was that if N3SP-1 inhibited CFTR activity, Isc would be significantly lower in loops exposed to CTx plus N3SP-1 compared to loops exposed to CTx in PBS or CP-1, as well as possibly demonstrating less subsequent stimulation of Isc by FSK or carbachol. In fact, the Isc in CTx treated loops exposed to CP-1 and N3SP-1 were slightly, but not significantly, greater than in the loops treated with CTx in PBS and there was no significant difference in Isc of the CTx plus CP-1 or NS3P-1 loops (FIG. 9A). In addition, the peak increases in Isc in CTx exposed loops with sequential addition of FSK and carbachol were not different between the CP-1 and N3SP-1 exposed loops (FIG. 9B). These results support that N3SP-1 does not inhibit CTx-induced Cl-secretion mediated by either cAMP or Ca²⁺ signaling but rather that N3SP-1 effects involve stimulation of Na⁺ absorption. We next determined whether this beneficial effect occurs in other murine diarrheal disease models.

E. coli heat stable enterotoxin (STa)-induced mouse jejunal fluid accumulation, a cGMP dependent effect (Sunuwar et al., 2020; Foulke-Abel et al., 2020), was studied. E. coli heat stable enterotoxin (0.5 μg purified STa) caused intestinal fluid accumulation that was significantly reduced by N3SP-1 (FIG. 3C). This effect was not observed when STa-treated loops were exposed to CP-1. Thus, N3SP-1 not only stimulates basal NHE3 activity and net jejunal water absorption, but also prevents changes in net water secretion caused by two major bacterial enterotoxins responsible for cholera and Traveler's diarrhea.

We next determined whether N3SP-1 exhibits similar effects in a previously standardized diarrheal model of TNFα-mediated inflammation (via anti-CD3 monoclonal antibodies) (Clayburgh, 2006). Mouse jejunal loops were prepared containing 100-μL PBS with either 400-nM CP-1 or N3SP-1. Following surgery, 200-μL PBS alone or with 200-μg anti-CD3 antibody were injected IP and mice sacrificed 2.5 h later. Anti-CD3 antibody increased luminal fluid accumulation in CP-1 containing loops, but not in NS3P-1 loops (FIG. 3D). This observation demonstrates that N3SP-1 also prevents fluid secretion in an inflammatory diarrhea model. This effect was not specific for sex (FIG. 9C).

Since in silico modeling of N3SP-1 suggested a potential interaction between rabbit R575 and D602, we determined whether R575 was necessary for N3SP-1 effects. Purified peptides were synthesized that lack either the first seven (N3SP-1Δ7) or ten (CP-2)N-terminal amino acids of N3SP-1, where only N3SP-1Δ7 contains R575 (FIG. 1B, FIG. 8A). These peptides were tested in CTx loops, except that 400 nM CP-2 and N3SP-1Δ7 were used (FIG. 3E). Results similar to those with N3SP-1 and CP-1 were found supporting that deletion of the N-terminal 7 amino acids of N3SP-1 did not alter the efficacy of this peptide to reduce CTx-related diarrhea.

1.4.2.3 Nanoparticle Delivery of N3SP-1 into ex vivo Human Jejunal Enteroid Monolayers

Because maleimide conjugation does not represent a viable delivery system for human use, nanoparticle delivery of CP-2 and N3SP-1Δ7 was tested in human jejunal enteroid monolayers. Polymeric nanoparticles comprised of hyperbranched carboxylated poly(beta-amino) ester (cPBAE) with carboxylate ligand endcaps were selected (Kozielski et al., 2013; Rui et al., 2019; Wilson et al., 2019). (FIG. 10A). This class of polymers enables efficient cellular uptake, endosomal escape, and cytosolic delivery of various proteins ranging from 27 kDa to 160 kDa in size (Rui et al., 2019). Hyperbranched PBAE polymer is advantageous as a biomaterial for intracellular delivery due to its pH-responsiveness, low cytotoxicity, and biodegradability (Wilson et al., 2019). Further, the polymer used for this study, termed CR5 (FIG. 10A), incorporates bio-reducible disulfide bonds in the polymer backbone, which facilitates efficient triggered release of cargo preferentially in the cytosol, where glutathione concentrations are significantly elevated relative to the extracellular environment (Chen et al., 2015). Nanoparticle synthesis, characterization, and pH effects on toxicity and cellular uptake in the cancer cell line, B16-F10 are described in FIG. 10A-FIG. 10I.

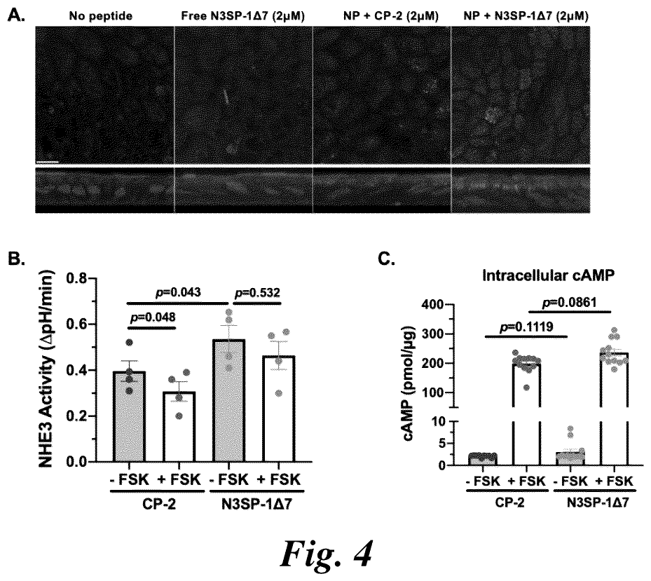

Given the effectiveness of N3SP-1Δ7 in preventing CTx-induced net fluid secretion in mouse intestine in vivo, enteroid studies were performed to demonstrate nanoparticle uptake into human intestinal epithelial cells. Optimized nanoparticle formulations containing either CP-2 or N3SP-1Δ7 were added apically to differentiated human jejunal enteroid monolayers. Fluorescently labeled nanoparticle location was identified by confocal microscopy and compared to endogenous apical NHE3. Nanoparticles appeared intracellularly confirming uptake in normal human intestinal epithelial cells (FIG. 4A). Similar studies were performed in B16-F10 cells, demonstrating time and concentration dependent uptake, while in the absence of nanoparticles, no significant uptake occurred (FIG. 10G).

For functional studies, we considered that in vitro delivery of nucleotides by PBAE nanoparticles peaked within 24-48 h of nanoparticle exposure (Rui et al., 2019). Thus, nanoparticles were added apically (250 nM) for 18 h to differentiated enteroid monolayers and the effect on basal and FSK-inhibited NHE3 activity was determined. NHE3 activity was significantly increased in N3SP-1Δ7 compared to CP-2 (increase of 36.0%+10.4%, n=4, p=0.043). FSK significantly reduced NHE3 activity in CP-2 exposed enteroids (77.2%+7.4% of control peptide, n=4, p=0.048) (FIG. 4B). In contrast, in N3SP-1Δ7 exposed enteroids, FSK caused a non-significant decrease in NHE3 activity (88.0%+6.7% of N3SP-1, n=4, p=0.170).

To further interrogate the loss of the FSK effect, it was determined whether the FSK stimulation of adenylate cyclase-cAMP was altered by N3SP-1Δ7. In jejunal enteroids exposed to FSK, increased intracellular cAMP was similar between CP-2 and N3SP-1Δ7 (FIG. 4C). This observation demonstrates that the prevention of FSK inhibition of NHE3 occurred downstream of FSK-stimulated adenylate cyclase-increased cAMP. Thus, as observed with maleimide-based delivery of N3SP-1, nanoparticle delivery in human enteroids stimulated basal NHE3 activity and prevented CAMP inhibition of NHE3.

Given the ability of N3SP-1Δ7 to prevent CTx-induced fluid secretion in vivo (FIG. 3E) and to stimulate basal NHE3 activity and prevent cAMP inhibition in human enteroids (FIG. 4C), further studies were carried out with human enteroids to determine the shortest N3SP-1-related peptide that would stimulate human NHE3. A series of N3SP-1 N-terminal truncations was synthesized and concentration-dependent stimulation of NHE3 activity determined using human jejunal enteroids. As shown in Table 2, deleting 5, 6, or 7 aa from the N3SP-1 N-terminus produced peptides that stimulated NHE3 similarly to full-length N3SP-1 both in magnitude of the stimulation and EC₅₀. Removing 8 aa, however, significantly reduced the maximum stimulatory effect and increased the EC₅₀, while removing more than 8 aa totally prevented NHE3 stimulation.

### 1.4.2.4 In Vivo Studies-Nanoparticle Conjugates and Intestinal Fluid Absorption

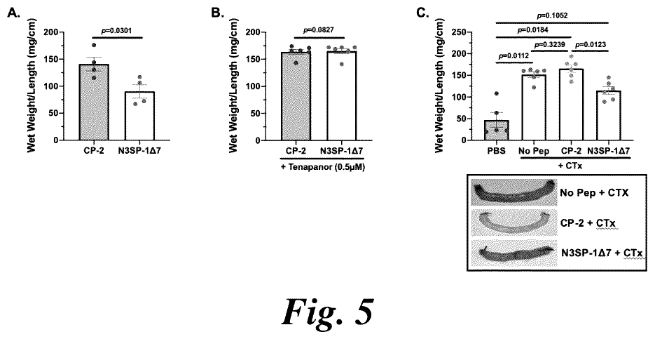

To treat acute diarrhea, the drug effect should have a rapid onset. Consequently, the effects of the nanoparticle conjugated CP-2 compared to N3SP-1Δ7 was determined on net fluid transport in closed 3-4 cm ileal loops exposed to 400-nM peptide for 4 h followed by draining of the loops and exposure to 150-μL PBS for 30 min. As shown in FIG. 5A, N3SP-147 incubation produced significantly less amount of residual fluid, consistent with increased fluid absorption. To demonstrate that this observation was due to increased Na⁺ absorption caused by N3SP-1Δ7, these closed loop studies were repeated with the specific NHE3 inhibitor (Spencer et al., 2014) Tenapanor (10 μM), present during both incubation periods. In the presence of Tenapanor, there was no difference in residual fluid comparing CP2 and N3SP-1Δ7 (compare FIG. 5A and FIG. B).

An additional study was performed to determine if nanoparticle-N3SP pretreatment could prevent cholera toxin-induced fluid secretion. Nanoparticle-N3SP-1Δ7 or -CP2 (4 μM) with 0.1-μg CTx in 150-μL PBS were inoculated into closed mouse ileal loops as above. Four hours later the animals were sacrificed and loop weight/length determined. Loops only injected with cholera toxin and PBS and PBS alone with the same total volume as the nanoparticle injected loops were studied as additional controls. As shown in FIG. 5C, NS3P-147 loops had significantly less fluid at 4 hours than the other two conditions. CP2 did not reduce the residual loop volume. Subtracting the PBS only loop weight/length from the cholera toxin incubated loops indicated the NS3P-147 reduced the cholera toxin induced secretion by approximately 42%. This result demonstrates that, as with the BODIPY-N3SP studies, nanoparticle-N3SP-1Δ7 reduced cholera toxin-induced intestinal fluid secretion.

### 1.4.2.5 NHE3 Peptide-NHERF2 Overlay Experiments: Effects of CP2 and N3SP-1Δ7 E3 Peptides

To further test the hypothesis that N3SP-1 stimulated NHE3 by competing for binding with components of the IRCX, overlay experiments were performed examining binding of NHERF2 to NHE3 peptides. NHERF2 is a two PDZ domain containing scaffold that is involved in intestinal NHE3 stimulation and inhibition (Donowitz and Li, 2007; Donowitz et al., 2009; Sarker et al., 2008; Akhter et al., 2002; Murtazina et al., 2011.

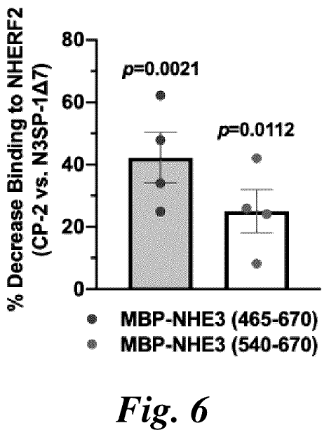

Binding of purified full-length GST-NHERF2 to two NHE3 C-terminal peptides that contain the N3SP-1 sequence was determined in the presence of equal concentrations of CP-2 compared to N3SP-1Δ7. CP-2 and N3SP-1Δ7 peptides were used at 5 times the concentration of the NHERF2 fusion protein and the MBP-NHE3 fusion peptides. N3SP-147 significantly reduced NHERF2 binding compared to the control peptide (FIG. 6).

### 1.5 Discussion

This study strongly supports NHE3 as a druggable target for treatment of diarrheal diseases in which NHE3 is present and is inhibited. NHE3 is inhibited in almost all diarrheal diseases, including secretory and inflammatory diarrheal diseases (Zachos et al., 2005; Sarker et al., 2008; Akhter et al., 2002). In fact, while stimulated anion secretion is considered to be the major contributor to volume loss in secretory diarrheal diseases, such as cholera and Traveler's Diarrhea, this does not appear to be the case for inflammatory diarrheal diseases, including inflammatory bowel diseases or radiation enteritis, among others. Camilleri et al., 2017; Barrett 2020.

This example describe a peptide that stimulates basal NHE3 activity and prevents NHE3 inhibition caused by elevated cAMP, cGMP, and Ca²⁺, second messengers known to inhibit NHE3 and to be intermediates in human diarrheal diseases. Multiple intestinal models were studied that included both in vivo and in vitro mouse intestine, as well as in vitro cell lines including fibroblasts expressing NHE3, the polarized human colon cancer cell line Caco-2/BBe, and ex vitro normal human jejunal enteroids (Levine et al., 1993; Sarker et al., 2008; Sunuwar et al., 2020; Foulke-Abel et al., 2020; Murtazina et al., 2011).

We used these cell/tissue/animals/human enteroids to model secretory and inflammatory diarrheal diseases, including two models of bacterial enterotoxin-induced secretion and a model of TNFα-related secretion. All models produced similar evidence of the effectiveness of NS3P to stimulate basal NHE3 activity and to reverse the inhibition that occurs in each of the diarrhea models studied. In addition, we determined that the efficacy of N3SP in stimulating NHE3 activity, which does not enter epithelial cells alone, required conjugation to either a cell-penetrating maleimide or to nanoparticles, since there was no effect without cell entry. Moreover, we showed that N3SP stimulated basal and second messenger inhibited NHE3 similarly at very low concentrations (EC₅₀ approximately 150 nM) using either delivery system.

Some insights have been provided from these studies concerning the mechanism(s) by which N3SP stimulates basal NHE3 activity and reverses NHE3 inhibition in the in vivo models of intestinal fluid secretion. Under normal conditions, NHE3 continually traffics between the endosomal system and the plasma membrane, with the percent on the BB determined by the balance between rates of endocytosis, exocytosis and BB protein stability (Zachos et al., 2016; Rao 2019; Zachos et al., 2005; Kapus et al., 1994; Sarker et al., 2008; Akhter et al., 2002).

The diarrhea related inhibition of NHE3 is usually due to abnormal trafficking caused by agents that induce the diarrhea, which act by elevating intracellular cAMP, cGMP, Ca²⁺, all of which stimulate rates of NHE3 endocytosis and often also inhibit exocytosis (Zachos et al., 2016; Rao, 2019; Zachos et al., 2005; Donowitz and Li, 2007; Donowitz et al., 2009; Donowitz et al., 2000; Chow et al., 1999; Chen et al., 2015; He et al., 2008. N3SP was engineered to mimic the intracellular NHE3 C-terminal domain that binds proteins to form the largest identified NHE3 regulatory complex, which is primarily involved in inhibiting NHE3 activity. This sequence of NHE3 is unique in the human genome and it allows effects specific for NHE3.

The proposed mechanism of N3SP stimulation of NHE3 is to compete with proteins that normally bind to the NHE3 C-terminus in the inhibitory domain and prevent their binding under basal conditions and with elevated second messenger signaling. Proteins shown to bind to this area of NHE3 and inhibit basal NHE3 activity include the kinase CaMKIIδ, CaM, PLCγ, and NHERFs 1-4 (Donowitz and Li, 2007; Donowitz et al., 2009; Sarker et al., 2008; Zizak et al., 2012; Zachos et al., 2009).

IRBIT-1, which also regulates NHE3 activity (He et al., 2008), binds just C-terminal of this area. That this is the mechanism involved in N3SP effects on NHE3 is supported by the reduced binding caused by N3SP compared to the control peptide of one of the major components of the NHE3 Inhibitory Complex (IRCX), NHERF2, to peptides of the NHE3 C-terminus that contain the N3SP sequence (FIG. 6). More detailed studies are required to identify all components of the IRCX and the effects of N3SP on their interactions with the NHE3 C-terminus, although that is beyond the scope of this study. The lack of structural information of the two NHE3 C-terminus fusion proteins prevents any insights on why effects of NS3P compared to control peptide were greater in competing with NHERF2 peptide in the larger NHE3 peptide.

Not understood is the important ability of N3SP-1 to completely prevent the fluid secretion induced in vivo by CTx, E. coli heat stable enterotoxin, and anti-CD3-induced inflammation. Enterotoxin-induced Cl-secretion, which is dependent on activation of CFTR, is thought to be the major component of cholera and Enterotoxigenic E. coli secretory diarrheas (Zachos et al., 2005; Camilleri et al., 2017; Barrett, 2020).

We were unable to identify any evidence that N3SP did other than stimulate NHE3; with the in vivo stimulation of NHE3 confirmed by the effect on basal transport being prevented by Tenapanor pretreatment (FIG. 5). N3SP-1 did not inhibit the FSK-stimulation of adenylate cyclase-cAMP in enteroids and did not alter the short-circuit current in CTx-exposed mouse jejunum or the subsequent stimulation induced sequentially by forskolin and carbachol, making an effect to inhibit stimulated Cl-secretion unlikely. Unlike CFTR, NHE3 has not been shown to be directly involved in regulation of other transport proteins. The only regulatory functions of NHE3 identified relate to its generation of an apical membrane H⁺ gradient, which provides some of the driving force of PEPT1 uptake of di-, tri-, and oligopeptides and some H⁺ gradient-linked amino acid transporters (Thwaites and Anderson, 2007; Watanabe et al., 2005) and separately stimulation of intestinal phosphate absorption, apparently by effects on specific tight junctional permeability to phosphate (Chen et al., 2015). Nonetheless, this is an area requiring further exploration testing the hypothesis that NHE3 is indirectly involved in regulation of other transport processes that contribute to fluid secretion in diarrheal diseases.

Structural information about the NHE3 C-terminus would allow us to understand why N3SP-1/N3SP-1Δ7 but not CP-1/CP-2 stimulates NHE3 activity. In spite of advances by cryoEM in understanding the structure of mammalian, as well as bacterial NHEs, there is very little experimental structural information concerning NHE C-termini distal to the proximal domain that binds Calcineurin Homologous Protein (CHP) that is adjacent to the transmembrane domain (Ben Ammar et al., 2005; Ammar et al., 2006; Pang et al., 2001; Sjogaard-Frich et al., 2021; Winklemann et al., 2020).

In addition, the recently reported remarkable ability of AlphaFold to define protein structure from amino acid sequences has been used to describe the structure of NHE3 (Tunyasuvunakool et al., 2021). This method predicted a structure of the NHE3 C-terminal domain similar to our Rosetta model; although this was in an area that the program described as “low confidence” (Tunyasuvunakool et al., 2021). The Rosetta structural model allowed us to engineer a peptide that stimulated NHE3 with a low EC₅₀ (approximately 150 nM). This model predicated that the stability of N3SP-1 was based on the peptide forming two alpha helixes which were stabilized by H⁺ bonding between human R576 and E602 (in rabbit R575 and D602). While structural stability has not been experimentally confirmed, truncation of N3SP-1 to form N3SP-1-NA7 (N terminal amino acid R575) preserved NHE3 stimulation with a similar EC₅₀ to N3SP-1 in enteroids, while efficacy was greatly reduced by removing one more N-terminal aa. This model predicts continued stability of the N3SP-1-NA7 by the same H⁺ bonding as N3SP-1, and that R575 at the beginning of the first helix in aa 575-605 is important for preserving functional ability of N3SP to stimulate NHE3 activity. This model also predicts decreased stability by removing R575, as occurs with the N3SP-1Δ8 peptide. Of note, the N-terminal region in CP-1 (residues 585-605) appears to form a flexible structure, supporting the assumption that a stable helical structure is important for the N3SP stimulation of NHE3 activity.

Considering the findings presented in this study together, we suggest that the NHE3 Stimulatory Peptide (N3SP) should be considered for further development as an anti-diarrheal drug. An important observation, was similar EC₅₀ for NHE3 stimulation by N3SP-1 in a human colon cancer line and normal human enteroids, which we have suggested as a potential way to assess potential human drug efficacy. Donowitz et al., 2020; Cil et al., 2017; Duan et al., 2019.

Nanoparticles were employed as a delivery vehicle for potential human use of N3SP. Nanoparticle delivery into the human colon cancer cell line, Caco-2 and rat small intestine has been demonstrated, and PBAE nanoparticles have been shown to deliver nucleic acids into HEK and B16-F10 cancer cells (Rui et al., 2019; Wilson et al., 2019). The peak delivery by PBAE nanoparticles of nucleic acids occurred 24-48 h after nanoparticle exposure (Rui et al., 2019). Mimicking this application, our initial studies with nanoparticle delivery of N3SP-1Δ7 was after overnight exposure. We also demonstrated effects with administration for 4 hours. Until now, we have not pursued oral delivery of N3SP-1Δ7 or considered issues of acid stability to pass through the stomach or release at specific segments of the small intestine or colon, which are the sites responsible for most diarrheal diseases. These considerations, however, have been dealt with for peptide therapies, such as linaclotide and plenalotide, and for numerous other drugs, such as budesonide; and consequently, we consider these are the next stage of development of N3SP-1Δ7 as a drug. In addition, further potential uses of N3SP-1Δ7 include as a tool to identify the components of the signaling complexes that regulate NHE3, and for further mechanistic studies to define the role of N3SP as an anti-diarrheal drug. Importantly, the efficacy of NS3P should be tested in additional diarrhea models, to define the range of potentially treatable diarrheas.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Zachos, N. C. et al. Human Enteroids/Colonoids and Intestinal Organoids Functionally Recapitulate Normal Intestinal Physiology and Pathophysiology. J Biol Chem 291, 3759-3766, doi: 10.1074/jbc.R114.635995 (2016).
Rao, M. C. Physiology of Electrolyte Transport in the Gut: Implications for Disease. Compr Physiol 9, 947-1023, doi: 10.1002/cphy.c180011 (2019).
Zachos, N. C., Tse, M. & Donowitz, M. Molecular physiology of intestinal Na⁺/H⁺ exchange. Annu Rev Physiol 67, 411-443, doi: 10.1146/annurev.physiol.67.031103.153004 (2005).
Walker, N. M. et al. Down-regulated in adenoma Cl/HCO₃ exchanger couples with Na/H exchanger 3 for NaCl absorption in murine small intestine. Gastroenterology 135, 1645-1653 e1643, doi: 10.1053/j.gastro.2008.07.083 (2008).
Xia, W. et al. The distinct roles of anion transporters Slc26a3 (DRA) and Slc26a6 (PAT-1) in fluid and electrolyte absorption in the murine small intestine. Pflugers Arch 466, 1541-1556, doi: 10.1007/s00424-013-1381-2 (2014).
Musch, M. W., Arvans, D. L., Wu, G. D. & Chang, E. B. Functional coupling of the downregulated in adenoma Cl/base exchanger DRA and the apical Na⁺/H⁺ exchangers NHE2 and NHE3. Am J Physiol Gastrointest Liver Physiol 296, G202-210, doi: 10.1152/ajpgi.90350.2008. (2009).
Haggie, P. M. et al. SLC26A3 inhibitor identified in small molecule screen blocks colonic fluid absorption and reduces constipation. JCI Insight 3, doi: 10.1172/jci.insight. 121370 (2018).
Kapus, A., Grinstein, S., Wasan, S., Kandasamy, R. & Orlowski, J. Functional characterization of three isoforms of the Na⁺/H⁺ exchanger stably expressed in Chinese hamster ovary cells. ATP dependence, osmotic sensitivity, and role in cell proliferation. J Biol Chem 269, 23544-23552 (1994).
Levine, S. A., Montrose, M. H., Tse, C. M. & Donowitz, M. Kinetics and regulation of three cloned mammalian Na⁺/H⁺ exchangers stably expressed in a fibroblast cell line. J Biol Chem 268, 25527-25535 (1993).
Camilleri, M., Sellin, J. H. & Barrett, K. E. Pathophysiology, Evaluation, and Management of Chronic Watery Diarrhea. Gastroenterology 152, 515-532 e512, doi: 10.1053/j.gastro.2016.10.014 (2017).
Barrett, K. E. Epithelial transport in digestive diseases: mice, monolayers, and mechanisms. Am J Physiol Cell Physiol 318, C1136-C1143, doi: 10.1152/ajpcell.00015.2020 (2020).
Donowitz, M. & Li, X. Regulatory binding partners and complexes of NHE3. Physiol Rev 87, 825-872, doi: 10.1152/physrev.00030.2006 (2007).
Donowitz, M. et al. NHE3 regulatory complexes. J Exp Biol 212, 1638-1646, doi: 10.1242/jeb.028605 (2009).
Sarker, R. et al. Casein kinase 2 binds to the C terminus of Na⁺/H⁺ exchanger 3 (NHE3) and stimulates NHE3 basal activity by phosphorylating a separate site in NHE3. Mol Biol Cell 19, 3859-3870, doi: 10.1091/mbc.E08-01-0019 (2008).
Akhter, S. et al. Na (+)/H (+) exchanger 3 is in large complexes in the center of the apical surface of proximal tubule-derived OK cells. Am J Physiol Cell Physiol 283, C927-940, doi: 10.1152/ajpcell.00613.2001 (2002).
Hendus-Altenburger, R., Kragelund, B. B. & Pedersen, S. F. Structural dynamics and regulation of the mammalian SLC9A family of Na (+)/H (+) exchangers. Curr Top Membr 73, 69-148, doi: 10.1016/B978-0-12-800223-0.00002-5 (2014).
Zizak, M. et al. Calmodulin kinase II constitutively binds, phosphorylates, and inhibits brush border Na⁺/H⁺ exchanger 3 (NHE3) by a NHERF2 protein-dependent process. J Biol Chem 287, 13442-13456, doi: 10.1074/jbc.M111.307256 (2012).
Zachos, N. C. et al. Phospholipase C-gamma binds directly to the Na⁺/H⁺ exchanger 3 and is required for calcium regulation of exchange activity. J Biol Chem 284, 19437-19444, doi: 10.1074/jbc.M109.006098 (2009).

Boehning D. et al. A peptide inhibitor of cytochrome c/inositol 1,4,5-trisphosphate receptor binding blocks intrinsic and extrinsic cell death pathways. Proc Natl Acad Sci U S A. 102, 1466-71. doi: 10.1073/pnas.0409650102 (2005).
Bender, B. J. et al. Protocols for Molecular Modeling with Rosetta3 and RosettaScripts. Biochemistry 55, 4748-4763, doi: 10.1021/acs.biochem.6b00444 (2016).
Rohl, C. A., Strauss, C. E., Misura, K. M. & Baker, D. Protein structure prediction using Rosetta. Methods Enzymol 383, 66-93, doi: 10.1016/S0076-6879 (04) 83004-0 (2004).
Bonneau, R. et al. De novo prediction of three-dimensional structures for major protein families. J Mol Biol 322, 65-78, doi: 10.1016/s0022-2836 (02) 00698-8 (2002).
Clayburgh, D. R., Musch, M. W., Leitges, M., Fu, Y. X. & Turner, J. R. Coordinated epithelial NHE3 inhibition and barrier dysfunction are required for TNF-mediated diarrhea in vivo. J Clin Invest 116, 2682-2694, doi: 10.1172/JCI29218 (2006).
Sunuwar, L. et al. Mechanical Stimuli Affect Escherichia coli Heat-Stable Enterotoxin-Cyclic GMP Signaling in a Human Enteroid Intestine-Chip Model. Infect Immun 88, doi: 10.1128/IAI.00866-19 (2020).
Foulke-Abel, J. et al. Phosphodiesterase 5 (PDE5) restricts intracellular cGMP accumulation during enterotoxigenic Escherichia coli infection. Gut Microbes 12, 1752125, doi: 10.1080/19490976.2020.1752125 (2020).
Kozielski, K. L., Tzeng, S. Y. & Green, J. J. A bioreducible linear poly(beta-amino ester) for siRNA delivery. Chem Commun (Camb) 49, 5319-5321, doi: 10.1039/c3cc40718g (2013).
Rui, Y. et al. Carboxylated branched poly(beta-amino ester) nanoparticles enable robust cytosolic protein delivery and CRISPR-Cas9 gene editing. Sci Adv 5, eaay3255, doi: 10.1126/sciadv.aay3255 (2019).
Wilson, D. R., Rui, Y., Siddiq, K., Routkevitch, D. & Green, J. J. Differentially Branched Ester Amine Quadpolymers with Amphiphilic and pH-Sensitive Properties for Efficient Plasmid DNA Delivery. Mol Pharm 16, 655-668, doi: 10.1021/acs.molpharmaceut.8b00963 (2019).
Spencer, A. G., Labonte, E. D., Rosenbaum, D. P., Plato, C. F., Carreras, C. W., Leadbetter, M. R., Kozuka, K., Kohler, J., Koo-McCoy, S., He, L., Bell, N., Tabora, J., Joly, K. M., Navre, M., Jacobs, J. W., Charmot, D. Intestinal inhibition of the Na⁺/H⁺ exchanger 3 prevents cardiorenal damage in rats and inhibits Na⁺ uptake in humans. Sci Transl Med 6:227ra36. doi: 0.1126/scitranslmed.3007790 (2014).
Murtazina, R. et al. NHERF2 is necessary for basal activity, second messenger inhibition, and LPA stimulation of NHE3 in mouse distal ileum. Am J Physiol Cell Physiol 301, C126-136, doi: 10.1152/ajpcell.00311.2010 (2011).
Yin, J. et al. Fluid shear stress enhances differentiation of jejunal human enteroids in Intestine-Chip. Am J Physiol Gastrointest Liver Physiol 320, G258-G271, doi: 10.1152/ajpgi.00282.2020 (2021).
Donowitz, M. et al. Short-term regulation of NHE3 by EGF and protein kinase C but not protein kinase A involves vesicle trafficking in epithelial cells and fibroblasts. Ann N Y Acad Sci 915, 30-42, doi: 10.1111/j.1749-6632.2000.tb05221.x (2000).
Chow, C. W., Khurana, S., Woodside, M., Grinstein, S. & Orlowski, J. The epithelial Na (+)/H (+) exchanger, NHE3, is internalized through a clathrin-mediated pathway. J Biol Chem 274, 37551-37558, doi: 10.1074/jbc.274.53.37551 (1999).
Chen, T. et al. Cyclic GMP kinase II (cGKII) inhibits NHE3 by altering its trafficking and phosphorylating NHE3 at three required sites: identification of a multifunctional phosphorylation site. J Biol Chem 290, 1952-1965, doi: 10.1074/jbc.M114.590174 (2015).
He, P., Zhang, H., Yun, C. C. IRBIT, inositol 1,4,5-triphosphate (IP3) receptor-binding protein released with IP3, binds Na⁺/H⁺ exchanger NHE3 and activates NHE3 activity in response to calcium. J Biol Chem. 83:33544-53. doi: 10.1074/jbc.M805534200. (2008)
Thwaites, D. T. & Anderson, C. M. H⁺-coupled nutrient, micronutrient and drug transporters in the mammalian small intestine. Exp Physiol 92, 603-619, doi: 10.1113/expphysiol.2005.029959 (2007).
Watanabe, C. et al. Na⁺/H⁺ exchanger 3 affects transport property of H⁺/oligopeptide transporter 1. Drug Metab Pharmacokinet 20, 443-451, doi: 10.2133/dmpk.20.443 (2005).
King, A. J. et al. Inhibition of sodium/hydrogen exchanger 3 in the gastrointestinal tract by tenapanor reduces paracellular phosphate permeability. Sci Transl Med 10, doi: 10.1126/scitranslmed.aam6474 (2018).
Ben Ammar, Y. et al. Crystallization and preliminary crystallographic analysis of the human calcincurin homologous protein CHP2 bound to the cytoplasmic region of the Na⁺/H⁺ exchanger NHE1. Acta Crystallogr Sect F Struct Biol Cryst Commun 61, 956-958, doi: 10.1107/S1744309105030836 (2005).
Ammar, Y. B., Takeda, S., Hisamitsu, T., Mori, H. & Wakabayashi, S. Crystal structure of CHP2 complexed with NHE1-cytosolic region and an implication for pH regulation. EMBO J 25, 2315-2325, doi: 10.1038/sj.emboj.7601145 (2006).
Pang, T., Su, X., Wakabayashi, S. & Shigekawa, M. Calcineurin homologous protein as an essential cofactor for Na⁺/H⁺ exchangers. J Biol Chem 276, 17367-17372, doi: 10.1074/jbc.M100296200 (2001).
Sjogaard-Frich, L. M. et al. Dynamic Na (+)/H (+) exchanger 1 (NHE1)-calmodulin complexes of varying stoichiometry and structure regulate Ca (2+)-dependent NHE1 activation. Elife 10, doi: 10.7554/eLife.60889 (2021).
Winklemann, I. et al. Structure and elevator mechanism of the mammalian sodium/proton exchanger NHE9. EMBO J 39, e105908, doi: 10.15252/embj.2020105908 (2020).
Tunyasuvunakool, K, et al. Highly accurate protein structure prediction for the human proteome. Nature 596, 590-596, doi: org/1038/s41586-021-03828-1 (2021)
Donowitz, M., Turner, J. R., Verkman, A. S. & Zachos, N. C. Current and potential future applications of human stem cell models in drug development. J Clin Invest 130, 3342-3344, doi: 10.1172/JCI138645 (2020).
Cil, O. et al. Benzopyrimido-pyrrolo-oxazine-dione CFTR inhibitor (R)-BPO-27 for antisecretory therapy of diarrheas caused by bacterial enterotoxins. FASEB J 31, 751-760, doi: 10.1096/fj.201600891R (2017).
Duan, T. et al. Inhibition of CFTR-mediated intestinal chloride secretion as potential therapy for bile acid diarrhea. FASEB J 33, 10924-10934, doi: 10.1096/fj.201901166R (2019).
Tse, C. M. et al. Enterohemorrhagic E. coli (EHEC)-Secreted Serine Protease EspP Stimulates Electrogenic Ion Transport in Human Colonoid Monolayers. Toxins (Basel) 10, doi: 10.3390/toxins 10090351 (2018).
Noel G N et al. A primary human macrophage-enteroid co-culture model to investigate mucosal gut physiology and host-pathogen interactions. Nature Scientific Reports. 7, 45270, doi: 10.1038/srep45270 (2017).
Staab J F et al. Co-culture system of human enteroids/colonoids with innate immune cells (i.e. macrophages and neutrophils). Current Protocols in Immunology. 131: e113, doi: 10.1002/cpim.113 (2020).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A peptide having at least about 80% identity with an amino acid sequence selected from SEQ ID NO:1, SEQ ID NO:2, SEQ ID NO:3, and SEQ ID NO:4, or a fragment thereof.

2. The peptide of claim 1, having at least about 81, 82, 83, 84, 85, 86, 87, 88, 89, 90, 91, 92, 93, 94, 95, 96, 97, 98, 99 or 100% identity with an amino acid sequence selected from SEQ ID NO: 1, SEQ ID NO:2, SEQ ID NO:3, and SEQ ID NO:4, or a fragment thereof.

3. The peptide of claim 1, wherein the peptide comprises SEQ ID NO: 1 or SEQ ID NO:2.

4. The peptide of claim 3, wherein the peptide comprises SEQ ID NO:2.

5. A conjugate of the peptide of claim 1, wherein the peptide is conjugated to a reporter molecule or a nanoparticle.

6. The conjugate of claim 5, wherein the reporter molecule comprises a hydrophobic reporter molecule.

7. The conjugate of claim 6, wherein the hydrophobic reporter molecule comprises a hydrophobic fluorescent maleimide.

8. The conjugate of claim 5, wherein the reporter molecule comprises 4,4-difluoro-4-bora-3a,4a-diaza-s-indacene (BODIPY) or an analog or derivative thereof.

9. The conjugate of claim 5, wherein the nanoparticle comprises one or more poly(beta-amino) esters (PBAEs).

10. The conjugate of claim 9, wherein the PBAE comprises a branched PBAE.

11. The conjugate of claim 10, wherein the branched PBAE comprises a carboxylated PBAE (cPBAE).

12. The conjugate of claim 10, wherein the branched PBAE comprises a compound of formula (I) or formula (II): 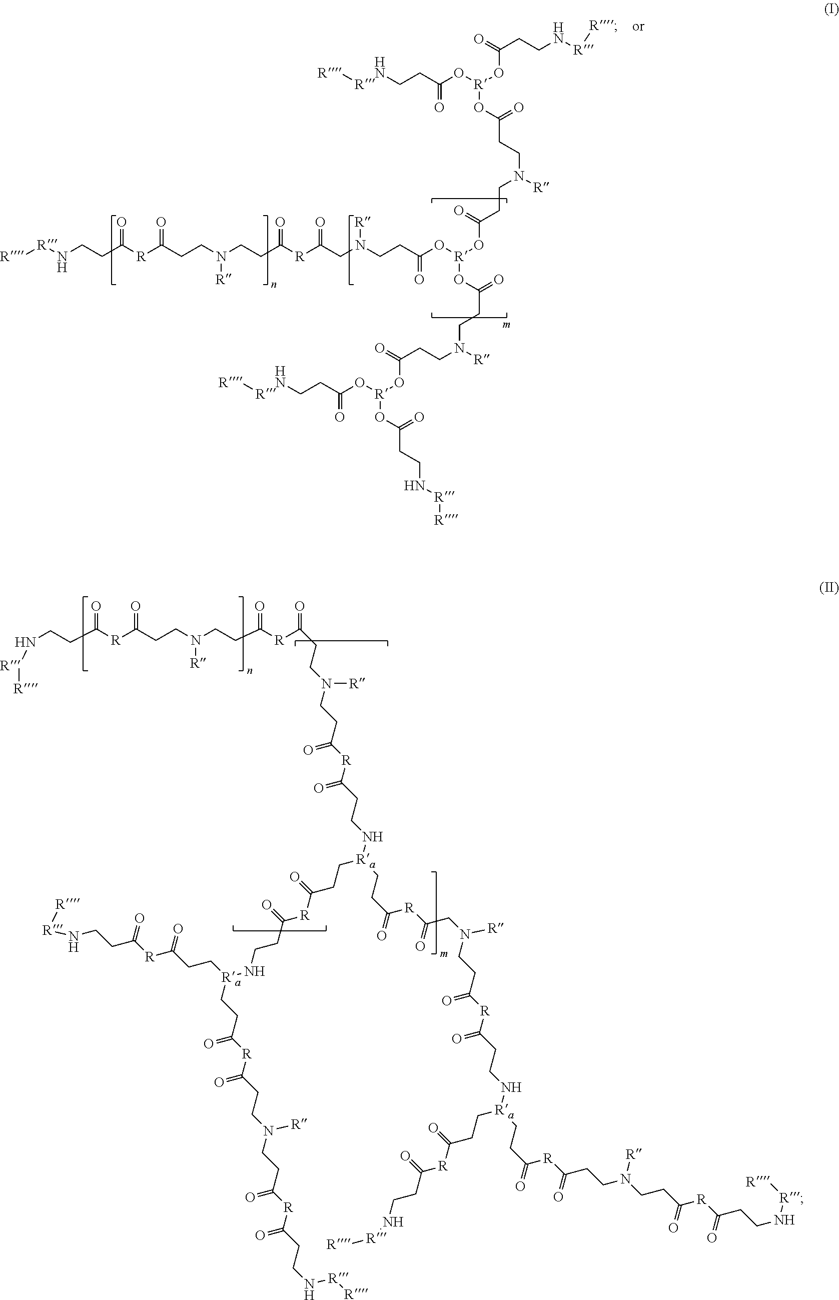
wherein: n and m are each independently an integer from 1 to 10,000; each R is independently a diacrylate monomer of the following structure: 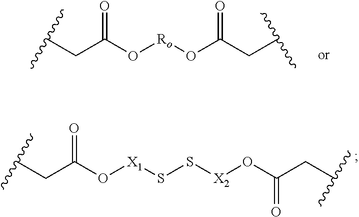
wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain; each R′ of formula (I) is a triacrylate, quanternary, or hexafunctional acrylate monomer selected from the group consisting of: 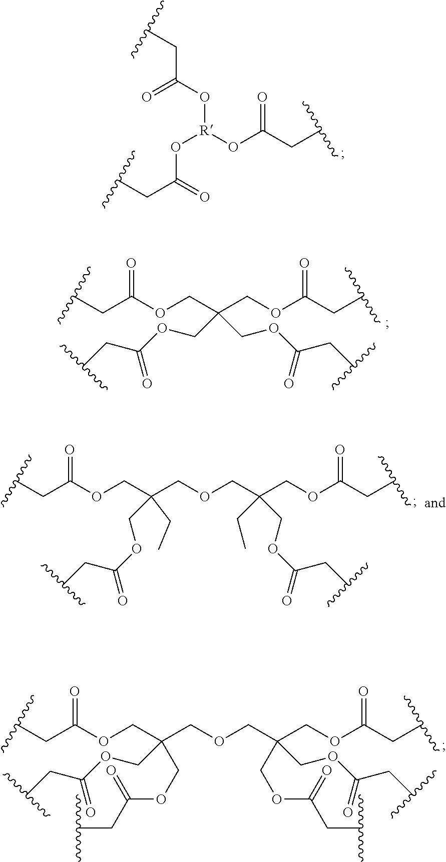
wherein each R′ is independently a trivalent group; each R″ is independently a side chain monomer comprising a primary, secondary, or tertiary amine; and each R′″ is independently an end group monomer comprising a primary, secondary, or tertiary amine.

13. The conjugate of claim 9, wherein the nanoparticle and the peptide have a weight-to-weight ratio of about 30:1 nanoparticle: peptide.

14. A method for treating a diarrheal disease, the method comprising administering to a subject in need of treatment thereof a therapeutically effective amount of the conjugate of claim 9.

15. The method of claim 14, the diarrheal disease comprises a diarrheal disease in which NHE3 is present and inhibited or in which NHE3 is present and not inhibited.

16. The method of claim 14, wherein the diarrheal disease is selected from an acute, a persistent, and a chronic diarrheal disease.

17. The method of claim 14, wherein administering a therapeutically effective amount of the peptide;
(a) treats one or more symptoms of the diarrheal disease;
(b) shortens a duration of the diarrheal disease, lessens a volume of diarrhea, and combinations thereof;
(c) increases intestinal Na⁺ absorption, rehydrates the subject, and combinations thereof;
(d) stimulates sodium-hydrogen exchanger 3 (NHE3) activity, prevents or diminishes CAMP inhibition of NHE3 activity, reverses reduced NHE3 activity caused by elevated cAMP, cGMP, and Ca²⁺, and inflammatory causes of diarrhea, and combinations thereof; and/or
(e) stimulates sodium-hydrogen exchanger 3 (NHE3) activity, prevents or diminishes CAMP inhibition of NHE3 activity, reverses reduced NHE3 activity caused by elevated cAMP, cGMP, and Ca²⁺, and inflammatory causes of diarrhea, and combinations thereof, in a small intestinal loop of the subject.

18.-21. (canceled)

22. The method of claim 17, wherein the small intestinal loop of the subject includes a jejunum and/or an ileum of the subject.

23. The method of claim 13, wherein the conjugate is delivered to a gastrointestinal tract (GI) tract of the subject.

24. The method of claim 23, wherein the conjugate is deposited subapically in the GI tract.

25. The method of claim 14, wherein the subject is an infant.

26. The method of claim 14, wherein the diarrheal disease is selected from watery diarrhea, fatty (malabsorption) diarrhea, inflammatory diarrhea, traveler's diarrhea, and diarrhea related to cholera.

27. (canceled)


SEQUENCE LISTING (9 sequences)

SEQ ID NO: 1 - protein, 38 aa, Synthetic construct
NVDFSTPRPSTVEASVSYLLRESASAVCLDMQSLEQRR

SEQ ID NO: 2 - protein, 31 aa, Synthetic construct
RPSTVEASVSYLLRESASAVCLDMQSLEQRR

SEQ ID NO: 3 - protein, 32 aa, Synthetic construct
PRPSTVEASVSYLLRESASAVCLDMQSLEQRR

SEQ ID NO: 4 - protein, 33 aa, Synthetic construct
TPRPSTVEASVSYLLRESASAVCLDMQSLEQRR

SEQ ID NO: 5 - protein, 21 aa, Synthetic construct
YLLRESASAVCLDMQSLEQRR

SEQ ID NO: 6 - protein, 28 aa, Synthetic construct
TVEASVSYLLRESASAVCLDMQSLEQRR

SEQ ID NO: 7 - protein, 37 aa, Synthetic construct
NVDFTPRSSTVEASVSYLLRENVSAVCLDMQSELQRR

SEQ ID NO: 8 - protein, 38 aa, Synthetic construct
NVDFSTPRPSTVEASVSYFLRENVSAVCLDMQSLEQRR

SEQ ID NO: 9 - protein, 38 aa, Synthetic construct
NVDFNTPRPSTVEASVSYFLRENVSAVCLDMQSLEQRR